(19) United States
(12) Patent Application Publication
(10) Pub. No.: US 2024/0360482 A1
(43) Pub. Date: Oct. 31, 2024

(54) TWO-STAGE ANAEROBIC DIGESTION PROCESSES AND SYSTEMS COMPRISING AMMONIA REMOVAL FOR CONVERTING ORGANIC MATERIAL INTO BIOGAS

(71) Applicant: CH FOUR BIOGAS INC., Smith Falls (CA)

(72) Inventors: Niloofar ABDEHAGH, Ottawa (CA); Benjamin STREHLER, Smiths Falls (CA); Majid SARTAJ, Ottawa (CA); Mohamad ADGHIM, Ottawa (CA)

(73) Assignee: CH FOUR BIOGAS INC., Smith Falls (CA)

(21) Appl. No.: 18/646,040

(22) Filed: Apr. 25, 2024

Related U.S. Application Data
(60) Provisional application No. 63/498,129, filed on Apr. 25, 2023.

(30) Foreign Application Priority Data
May 25, 2023 (CA) 3200837

(51) Int. Cl. C12P 5/02; C12M 1/107

(52) U.S. Cl. CPC C12P 5/023; C12M 21/04

(57) ABSTRACT

Described are two-stage anaerobic digestion processes and systems that are coupled with post-hydrolysis ammonia removal for digestion of high-nitrogen feedstock into biogas such as biomethane. The processes and related systems comprise a first-stage anaerobic digestion unit to obtain hydrolyzed material; a NH₃-stripping unit to obtain an ammonia-stripped slurry; and a second-stage anaerobic digestion unit for the methanogenesis of the ammonia-stripped slurry and production of a digestate and biogas. One process and system is adapted for operation in a continuous or semi-continuous mode. Another process and system comprise stripping with upgraded biogas.

### CROSS REFERENCE TO RELATED APPLICATIONS

The present application claims priority to US patent application No. 63/498,129 (filed on Apr. 25, 2023) and to Canadian patent application No. 3,200,837 (filed on May 25, 2023), the content of which is incorporated herein by reference in their entirety.

### FIELD OF THE INVENTION

The invention relates to the field of biogas generation from organic materials, and more particularly to processes and systems for digestion of high-nitrogen feedstock into biogas.

### BACKGROUND OF THE INVENTION

Anaerobic digestion (AD) of organic waste is a viable technology that can reduce the carbon footprint and greenhouse gas emissions of agricultural activities and other organic waste management techniques in addition to generating revenues by selling the produced biogas and digestate.

Ammonia stripping is an effective and economic technology to remove nitrogen from organic waste feedstock (Huang et al., 2019; Walker et al., 2011; Zhang et al., 2012). The technology relies on increasing the volatility of ammonia by increasing the pH and/or the temperature of the material and introducing a carrier gas to take the ammonia out of the system (Abouelenien et al., 2010; Adghim et al., 2021; Zhang et al., 2012). The method was proven to achieve high ammonia removal efficiencies of more than 80%, accompanied by an improvement in methane production that could reach up to 200% (Adghim et al., 2021; Zhang et al., 2012, 2017). Ammonia stripping also promotes nutrient recovery from the feedstock by capturing the ammonia from the carrier gas using scrubbers or traps such as sulfuric acid, forming high-grade fertilizers that can be used for agricultural purposes (Fuchs et al., 2018).

Post-hydrolysis ammonia stripping, sometimes referred to as pre-digestion ammonia stripping, was suggested and modeled by Walker et al. (2011). In their theoretical model, they suggested ammonia stripping at an intermediate location between the hydrolyzer and the main digester. More recently, a study showed that co-digestion, hydrolysis, and post-hydrolysis ammonia stripping can be combined to alleviate the inhibitory effect of ammonia and enhance the methane potential (Adghim M. et al., Waste and Biomass Valorization (2021) 12:6045-6056).

It has been proposed also to improve methane potential using stripping mediums such as air (Huang et al., 2019; Li et al., 2018) or using biogas (Nielsen et al., 2013; Serna-Maza et al., 2014). However, positive impact of impact of stripping with RNG on the methane potential has never been demonstrated.

Although the concepts of ammonia stripping have been described, research is still undergoing to optimize its operating conditions and utilization in AD applications.

Accordingly, there is a need for improved processes for anaerobic digestion of organic waste with high content (e.g., >3000 mg/L) of nitrogen. There is particularly a need for improved processes that can operate in a continuous or semi-continuous mode.

There is particularly a need for more efficient post-hydrolysis ammonia stripping of poultry manure in wet AD applications (10% TS).

There is also a need for optimization of post-hydrolysis ammonia stripping.

There is also a need to find an alternative stripping medium that is effective, anaerobic, and readily available in biogas plants.

There is also a need for higher methane production and for alleviating ammonia inhibition, under significantly less severe operating conditions.

There is particularly a need for processes and system wherein the stripping is carried out with upgraded biogas instead of air or regular biogas.

The present invention addresses these needs and other needs as it will be apparent from the review of the disclosure and description of the features of the invention hereinafter.

### BRIEF SUMMARY OF THE INVENTION

The invention is concerned with processes and systems for digestion of high-nitrogen feedstock into biogas.

According to one particular aspect, the invention relates to a continuous or semi-continuous two-stage anaerobic digestion process for converting organic material into biogas, comprising the steps of:
(1) hydrolysing the organic material under anaerobic conditions in a first-stage anaerobic digestion unit to obtain hydrolyzed material;
(2) stripping ammonia from the hydrolyzed material in a NH₃-stripping unit to obtain an ammonia-stripped slurry;
(3) proceeding to methanogenesis of the ammonia-stripped slurry in a second-stage anaerobic digestion unit to obtain a digestate and biogas;
wherein said process operates in a continuous or semi-continuous mode.

According to another particular aspect, the invention relates to a continuous or semi-continuous two-stage anaerobic digestion system for converting organic materials into biogas, comprising:
a first-stage anaerobic digestion unit configured to hydrolyse the organic material under anaerobic conditions and to obtain hydrolyzed material;
a NH₃-stripping unit configured to strip ammonia from the hydrolyzed material and obtain an ammonia-stripped digestate;
a second-stage anaerobic digestion unit configured for methanogenesis of the ammonia-stripped digestate and obtain a second digestate and a biogas comprising methane biogas;
wherein said system is adapted for operation in a continuous or semi-continuous mode.

According to another particular aspect, the invention relates to a two-stage anaerobic digestion process for converting organic materials into biogas, comprising the steps of:
(1) hydrolysing the organic material under anaerobic conditions in a first-stage anaerobic digestion unit to obtain hydrolyzed material;
(2) stripping ammonia from the hydrolyzed material in a NH₃-stripping unit to obtain an ammonia-stripped digestate, wherein said stripping comprises stripping with upgraded biogas;
(3) proceeding to methanogenesis of the ammonia-stripped digestate in a second-stage anaerobic digestion unit comprising to obtain a second digestate and a biogas comprising methane biogas.

According to another particular aspect, the invention relates to a two-stage anaerobic digestion system for converting organic materials into biogas, comprising:
a first-stage anaerobic digestion unit configured to hydrolyse the organic material under anaerobic conditions and obtain hydrolyzed material;
a NH₃-stripping unit configured for stripping ammonia from the hydrolyzed material and obtaining an ammonia-stripped digestate, wherein said NH₃-stripping unit is configured for stripping with upgraded biogas;
a second-stage anaerobic digestion unit configured for methanogenesis of the ammonia-stripped digestate and obtain a second digestate and a biogas.

According to another particular aspect, the invention relates to a two-stage anaerobic digestion process for converting organic materials into biogas, comprising the steps of:
(1) hydrolysing the organic material under anaerobic conditions in a first-stage anaerobic digestion unit to obtain hydrolyzed material;
(2) proceeding to methanogenesis of the hydrolyzed material in a second-stage anaerobic digestion unit to obtain a digestate and a biogas comprising methane biogas;
(3) stripping ammonia from the digestate in a NH₃-stripping unit to obtain an ammonia-stripped digestate, wherein said stripping comprises stripping with upgraded biogas.

According to another particular aspect, the invention relates to a two-stage anaerobic digestion system for converting organic materials into biogas, comprising:
a first-stage anaerobic digestion unit configured to hydrolyse the organic material under anaerobic conditions and obtain hydrolyzed material;
a second-stage anaerobic digestion unit configured for methanogenesis of the hydrolyzed material and obtain a second digestate and a biogas; and
a NH₃-stripping unit configured for stripping ammonia from the second digestate and for obtaining an ammonia-stripped digestate;
wherein said NH₃-stripping unit is further configured for stripping with upgraded biogas.

According to another particular aspect, the invention relates to a biogas plant comprising one or more two-stage anaerobic digestion system as defined herein.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE DRAWINGS

For the invention to be readily understood, embodiments of the invention are illustrated by way of example in the accompanying figures.

FIG. 1A is a diagram displaying a two-stage anaerobic digestion process, in accordance with one embodiment of the present invention.

FIG. 1B is a diagram displaying a two-stage anaerobic digestion process utilizing upgraded biogas, in accordance with one embodiment of the present invention.

FIG. 1C is a diagram displaying another two-stage anaerobic digestion process utilizing upgraded biogas, in accordance with one embodiment of the present invention.

FIG. 2 is diagram showing a semi-continuous two-stage anaerobic digestion coupled with post-hydrolysis ammonia removal system for converting organic materials into biogas, in accordance with one embodiment.

FIG. 3 is diagram showing a two-stage anaerobic digestion system for converting organic materials into biogas that is configured for stripping ammonia from hydrolyzed material using upgraded biogas, in accordance with one embodiment.

FIG. 4 is diagram showing side stripping of ammonia using upgraded biogas, in accordance with one embodiment. Stripping is performed on digested materials and returned into the digester to dilute ammonia levels.

FIG. 5 provides line graphs showing ammonia and PH levels during ammonia stripping of PM100 (a)), PM75:MS25 (b)), and PM50:MS50 (c)), in accordance with Example 1.

FIG. 6 provides line graphs showing net cumulative methane potential of untreated, hydrolyzed, and ammonia-stripped samples in preliminary batch mode testing, in accordance with Example 1.

FIG. 7 is a line graph displaying total ammonia (TKN) and total Kjeldahl (TKN), and daily TAN increase during hydrolysis, in accordance with Example 2.

FIGS. 8A-8B are line graphs displaying ammonia stripping results, in accordance with Example 2. FIG. 8A ammonia levels during the treatment, and FIG. 8B PH levels during the treatment.

FIGS. 9A-9D are line graphs showing impact of stripping on poultry manure's characteristics, in accordance with Example 2. FIG. 9A Chemical oxygen demand, FIG. 9B volatile fatty acids, FIG. 9C total alkalinity, and FIG. 9D total Kjeldahl nitrogen. Light grey bars represent before stripping and dark grey bars represent after stripping.

FIG. 10 is a line graph displaying net cumulative methane potential of ammonia-stripped, blended, and raw poultry manure, in accordance with Example 2.

FIGS. 11A and 11B are diagrams showing ammonia stripping configurations, in accordance with Example 3. FIG. 11A shows post-hydrolysis ammonia stripping; FIG. 11B shows side-stream ammonia stripping.

FIG. 12 provides dotted graphs displaying performance indicators of AD reactors, in accordance with Example 3. a) shows influent and effluent ammonia levels, b) shows volumetric biogas production, and c) shows specific biogas production.

FIG. 13 provides dotted graphs displaying performance conditions of anaerobic reactors, in accordance with Example 3. a) shows COD, b) shows VFA and alkalinity, c) shows pH, and d) shows solid contents.

Further details of the invention and its advantages will be apparent from the detailed description included below.

### DETAILED DESCRIPTION OF EMBODIMENTS

In the following description of the embodiments, references to the accompanying figures are illustrations of an example by which the invention may be practised. It will be understood that other embodiments may be made without departing from the scope of the invention disclosed. Unless defined otherwise, all technical and scientific terms used herein have the same meaning as commonly understood by one of ordinary skill in the art to which the invention belongs.

### General Overview

As described herein, the present invention is directed to two-stage anaerobic digestion processes and systems that are coupled with post-hydrolysis ammonia removal, and that are configured to operate in a continuous or semi-continuous mode. The present invention is also directed to two-stage anaerobic digestion processes and systems wherein upgraded biogas is used for stripping ammonium from hydrolyzed material.

As used herein the term “two-stage anaerobic digestion” refers to anaerobic digestion carried out in two separate steps or separate reactors, namely (i) a first-stage anaerobic digestion comprising hydrolysis of the organic materials, and (ii) a second-stage anaerobic digestion comprising methanogenesis of hydrolyzed organic materials.

As used herein the term “continuous” and “semi-continuous” are used interchangeably to refer to a mode of operation wherein organic material is fed, decanted and/or removed from the systems and/or processes on a regular basis to achieve a desired digestion target. This may include for instance feeding to, and/or drawing organic material from, the systems and/or processes on an ongoing or continuous basis and/or at intervals (e.g., one or more times per day or per week). In particular embodiments this may include feeding organic material to the first-stage anaerobic digestion unit and/or drawing therefrom hydrolyzed material. The term “continuous” and “semi-continuous” are used in opposition to the term “batch mode” wherein a given batch of organic material is processed, from beginning to end, with no or limited intervention during the process.

As used herein the term “organic material” broadly encompasses any organic material which can produce biogas by fermentation. This includes, but is not limited to, dairy manure, poultry manure, food wastes, corn silage and other agricultural residues, as well as municipal and industrial organic wastes. In embodiments the organic material comprises animal manure. In embodiments the animal manure is selected from poultry manure (PM), pig manure, cattle manure and mixtures thereof.

As used herein the term “biogas” broadly encompasses any organic gas, including gaseous fuel, especially methane, produced by the fermentation of organic matter. This term encompasses terms such as “upgraded biogas” and “RNG” (see hereinafter). Typically, biogas produced by fermentation comprises methane, carbon dioxide and other volatile compounds such as ammonia, hydrogen sulfide.

As used herein the term “upgraded biogas” refers to a biogas having a higher biomethane purity, such as but not limited to a biogas from which carbon dioxide has been removed. Typically, this result in an increased energy density since the concentration of methane is increased. The term upgraded biogas broadly encompasses, and sometime is used interchangeably with the term “renewable natural gas” or “RNG”. The term RNG refers to an upgraded biogas which may be obtained from a variety of sources, including municipal solid waste landfills, digesters at water resource recovery facilities (wastewater treatment plants), livestock farms, food production facilities and organic waste management operations. In embodiments, RNG includes biomethane.

### A) Process for Continuous or Semi-Continuous Two-Stage Anaerobic Digestion

One aspect of the invention concerns two-stage anaerobic digestion processes and systems conditions for converting organic materials into biogas. The two-stage anaerobic digestion processes and systems of the invention are advantageously coupled with post-hydrolysis ammonia removal, and they are configured to operate in a semi-continuous mode.

In embodiment the process is suitable for converting various types of organic materials including, but not limited to, animal manure (e.g., poultry manure (PM), pig manure, cattle manure and mixtures thereof), food wastes (e.g., cheese factory wastes, coffee-ground wastes, etc.), corn silage, agricultural residues (e.g., wheat straw, rice straw, sunflower shell, sugarcane bagasse), as well as municipal and industrial organic wastes (e.g., kitchen scraps, wastewater from pulp and paper mills), etc.

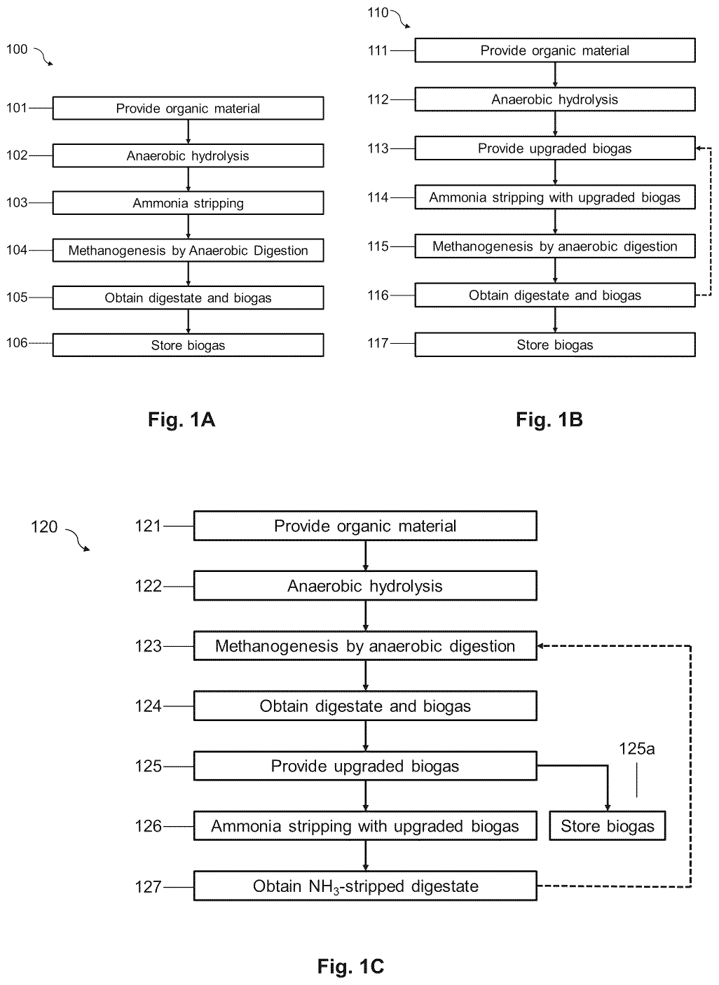

FIG. 1A depicts an example of such process. The two-stage anaerobic digestion process (100) for the continuous or semi-continuous conversion of organic materials into biogas comprises the steps of providing (101) organic material, hydrolysing (102) the organic material under anaerobic conditions in a first-stage anaerobic digestion unit to obtain hydrolyzed material, stripping (103) ammonia from the hydrolyzed material in a NH₃-stripping unit to obtain an ammonia-stripped slurry, proceeding to methanogenesis (104) of the ammonia-stripped slurry in a second-stage anaerobic digestion unit to obtain (105) a digestate and a desired biogas (e.g., methane). The biogas may be further stored (106), for later distribution and uses as a biofuel, and/or reuse in a digestion process as described hereinafter.

In embodiments, the process operates in a continuous or semi-continuous mode, i.e., it is possible to feed and/or withdraw materials from the process at any given point in time (e.g., continuous mode) or intermittently one or more times per day (e.g., semi-continuous mode).

FIG. 1B depicts an embodiment of a two-stage anaerobic digestion process (110) for the continuous or semi-continuous conversion of organic materials into biogas, the process utilizing upgraded biogas for ammonia stripping. The process (110) comprises providing (111) organic material, hydrolysing (112) the organic material under anaerobic conditions in a first-stage anaerobic digestion unit to obtain hydrolyzed material, providing (113) upgraded biogas, which biogas may at least partly be the result of methanogenesis (115), stripping (114) ammonia from the hydrolyzed material in a NH₃-stripping unit to obtain an ammonia-stripped hydrolyzed material, proceeding to methanogenesis (115) of the ammonia-stripped hydrolyzed material in a second-stage anaerobic digestion unit to obtain (116) a digestate and a desired biogas (e.g., methane). As illustrated by the dashed arrow, in this embodiment biogas obtained at (116) may be reused for ammonia stripping (113). The biogas may also be stored (117), for example for later distribution and uses, for example as fuel.

FIG. 1C depicts an embodiment of a two-stage anaerobic digestion process (120) for the continuous or semi-continuous conversion of organic materials into biogas, the process utilizing upgraded biogas for ammonia stripping. Process (120) comprises providing (121) organic material, hydrolysing (122) the organic material under anaerobic conditions in a first-stage anaerobic digestion unit to obtain hydrolyzed material, proceeding to methanogenesis (123) of the hydrolyzed material in a second-stage anaerobic digestion unit to obtain (124) a digestate and a desired biogas (e.g. methane), providing (125) upgraded biogas. The upgraded biogas may be stored (125a), for example for later distribution and uses, for example as fuel. Furthermore, in this embodiment the digestate obtained in the previous step (124) is stripped (126) in a NH₃-stripping unit with the upgraded biogas to obtain to obtain an ammonia-stripped digestate. As illustrated by the dashed arrow, the ammonia-stripped digestate may then be returned to the second-stage anaerobic digestion unit for further methanogenesis (123).

Additional details of processes (100,) (110) and (120) are described below. It will be understood that other useful variants and embodiments may be apparent to persons of skill practicing the present disclosure and are to be considered to fall within its scope.

### Anaerobic Hydrolysis (102, 112, 122)

Anaerobic hydrolysis comprises anaerobic hydrolysis of organic materials under anaerobic to obtain hydrolyzed material. This can be achieved in any suitable hydrolysis reactor such as a closed tank.

In embodiments the process is carried out in a completely closed tank at 40-45° C. with a retention time of five to ten days. The feedstock materials are mixed and during hydrolysis complex organic compounds (lipids, carbohydrate and proteins) molecules break down to simpler molecules (fatty acids, sugars and amino acids).

In embodiments, it may be advantageous to mix the material to ensure materials are properly mixed and/or to expedite the digestion process. It may also be advantageous to control the temperature to provide a temperature-controlled hydrolysis environment (e.g., about 40° C. to about 45° C. for the whole duration the hydrolysis).

In embodiments, the stripping comprises heating hydrolyzed material to be stripped (e.g., at about 55° C.) for about 3 to about 3.5 hours. In embodiments, the stripping is carried out at a temperature of about 40° C. to about 55° C. In one preferred embodiment, the stripping is carried out at a constant temperature of about 55° C.

In embodiments, the hydrolysis is carried out with raw material having a relatively low total solid (TS of 10-15%). Dilution using water, wash water, or low TS feedstock may be necessary if TS of raw feedstock is greater than 15%.

The process may further comprise an optional dilution or homogenization step. In accordance with the present invention, it may be advantageous to dilute and/or homogenize the organic material prior to and/or during anaerobic hydrolysis. This can be achieved for instance by mixing the organic material prior to and/or during the hydrolysis of step. Dilution may be required depending on the type of hydrolysis reactors being used. In continuously stirred tank reactors, dilution may be necessary to reduce total solids to at least 10-15% w/w. In plug-flow reactors, dilution may be necessary to reduce total solids to 20-25% w/w. If feedstock total solids concentration is already less than mentioned values, then dilution is typically not needed. Dilution material can consist of water, wash water in manure cleaning systems, liquid portion of digestate, or including a low-solid feedstock in the recipe such as sewage sludge.

The process may further comprise an optional solid-liquid separation step. This optional step is carried out before the stripping (103, 114, 126) and aims to separate a liquid portion and a solid portion from the hydrolyzed material obtained at step 102, 112 or 122. Therefore, only the liquid portion undergoes subsequent ammonia stripping treatment while the solid portion is added or returned to the hydrolysis step.

The solid-liquid separation step may be advantageous to reduce clogging in the NH₃-stripping unit. It may also provide the advantage of avoiding affecting the microorganisms responsible for biogas production (e.g., side-stream ammonia stripping). Indeed, it is preferable avoiding compromising microbial growth of methanogenic archaea as they are susceptible to shock changes to pH and temperature. Returning the solid portion to the hydrolysis step is also advantageous for diluting ammonia levels of the organic material being hydrolyzed.

### Ammonia Stripping (103, 114, 126)

Ammonia stripping comprises stripping ammonia from the hydrolyzed material to obtain an ammonia-stripped material, which may be a slurry or just a liquid portion of a hydrolyzed material or a digestate. Various devices may be acceptable for the stripping process. This may be achieved in a NH₃-stripping unit which provides for a gas-to-liquid transfer process wherein a gas (e.g., air, upgraded biogas or RNG) carries volatile ammonia as the gas travels through the hydrolyzed material. As such, various techniques or devices may be acceptable for the stripping step. For instance, if the carrier gas is air, upgraded biogas or RNG, the carrier gas flow rate may be set at about 100 L gas per L material per hour (e.g., about 50 to about 300 L gas per L material per hour). Stripping duration may be set to about 2.5 hours (about 2 to about 4 hours). Stripping duration may be determined by the ammonia removal efficiency decline rate. Stripping conditions may vary if ammonia levels change with time. For biogas stripping, the biogas flow rate may be below 100 L gas per L material, for example below 50, below 20, below 10 L biogas per L material per hour. Lower gas flow rates may be advantageous by at least partially preserving the material's buffering capacity. In biogas stripping, the pH may be basic, for example above pH 7, above pH 8, above pH 9, above pH 10. Biogas stripping may be carried out at suitable temperatures, for instance between 60° C. and 80° C., preferably between 65° C. and 75° C. The duration of the biogas stripping step may be any duration suitable to remove a predetermined proportion of ammonia, or to obtain a stripped material meeting predetermined parameters. It will be understood that longer stripping steps may yield a lower ammonia content. For example, the stripping step may last six hours. In some embodiments, the stripping step duration may exceed six hours.

In embodiments, the pH may be increased inside during the ammonia-stripping step to promote ammonia volatility. This can be achieved by providing an alkaline inflow during the ammonia-stripping step. The alkaline inflow may comprise any suitable alkaline compound including, but not limited to, lime, NaOH, etc. In embodiments the pH of the stripping is carried out at a pH of about 9.5 to about 10.0, preferably about pH 9.5. Alkaline materials may be used as solids, as liquids or as slurry.

In embodiments, ammonia stripping provides an ammonia-stripped slurry in which ammonia nitrogen has been reduced by at least 50%, or at least 60%, or at least 70%, or at least 75%, or at least 80%, compared to content of ammonia nitrogen present in the hydrolyzed material prior to the ammonia stripping. For instance, the hydrolyzed material may comprise an ammonia nitrogen content of about 2500 to about 6000 mg NH₃—N/L. Accordingly, with a 75% efficiency the ammonia-stripped digestate would comprise about 625 to about 1500 mg NH₃—N/L, and with a 80% efficiency the ammonia-stripped digestate would comprise about 500 to about 1200 mg NH₃—N/L. Preferably, the stripping is such that the ammonia-stripped slurry comprises less than about 3000 mg NH₃—N/L, or less than about 2750 mg NH₃—N/L, or less than about 2500 mg NH₃—N/L, or less than about 2250 mg NH₃—N/L, or less than about 2000 mg NH₃—N/L.

It is also within the skills of those in the art to find suitable stripping conditions and parameters, including materials, temperature, duration, etc. to produce an ammonia-stripped slurry suitable for the next steps of the process (e.g., methanogenesis).

The process may also further a NH₃-scrubbing step, for removing volatile ammonia carried by the gas that traveled through the hydrolyzed material during the step of ammonia stripping. In embodiments, the NH₃ scrubbing step comprises reacting gaseous ammonia with sulfuric acid to produce ammonium-sulfate and a gaseous effluent low in ammonia (i.e., a low-NH₃ effluent).

In embodiments the gaseous effluent low in ammonia is flared or simply discharged to atmosphere. This may be advantageous if air is used for the ammonia stripping and if ammonia levels are considered to be safe (e.g., according to federal, provincial, territorial and/or local regulations).

In embodiments the gaseous effluent low in ammonia is recirculated into the ammonia stripping unit. Recirculation may be particularly advantageous when biogas or RNG is used for the ammonia stripping since recirculation reduces the amount of fresh biogas or RNG needed for ammonia stripping and also because the gaseous effluent low in ammonia cannot be released to atmosphere without flaring.

Typically, the ammonium-sulfate produced during the NH₃-scrubbing unit will be a liquid effluent. The additional NH₃-scrubbing step may comprise discarding that liquid effluent, further processing the liquid effluent and/or using the liquid effluent as a high-quality fertilizer (i.e., nutrient-rich fertilizer).

### Methanogenesis (104, 115, 123)

Methanogenesis comprises proceeding to methanogenesis of the ammonia-stripped slurry in a second-stage anaerobic digestion unit to obtain a digestate and a biogas which typically comprises methane and other gaseous compounds such as carbon dioxide, sulfur and/or other impurities. In related steps, the biogas so is collected and optionally purified (e.g., to remove carbon dioxide, sulfur and/or other impurities) to produce upgraded biogas, compressed for storage (106, 117, 125a) and/or compressed for injection to a natural gas pipeline. Alternatively, in a related step, the biogas, upgraded or purified biogas, and RNG produced during methanogenesis is recirculated in the process for ammonia stripping.

In embodiments, the methanogenesis is carried out a constant temperature of about 37° C. to about 39° C. In embodiments, the step is carried out with a hydraulic retention time of about 25 to about 30 days.

Operating conditions of the methanogenesis are preferably favorable for methanogenic archaea. Accordingly, in preferred embodiment, the methanogenesis may comprise controlling pH and/or ammonia levels. Controlling the pH to an upper range suitable for anaerobic digestion (e.g., pH of about 7 to about 8) can help reducing competition of methanogenic archaea with other microorganisms such as sulfur-reducing bacteria over carbon. Likewise, a degree of ammonia removal may impact on distribution of methanogenic microorganisms amongst acetoclastic and hydrogenotrophic methanogens. Ideally, ammonia level should be low enough to promote the growth of both methanogens to maximize utilization of organic materials. pH may be adjusted through the addition of alkalis such as lime or soda ash, or acids such as phosphoric acid or nitric acid. In embodiments, ammonia level is controlled by the NH₃-stripping unit that achieves certain ammonia removal based on the operating conditions including, but not limited to, flowrate, pH, and temperature of the NH₃-stripping column.

In embodiments, the biogas exiting the second-stage anaerobic digestion unit comprises about 50 to 65% w/w methane, about 35 to 50% w/w carbon dioxide, and about 0 to 3% w/w of other gases (e.g., water vapour, hydrogen sulfide, ammonia, etc). In embodiments, biogas obtained and collected from the second-stage anaerobic digestion unit comprises at least 60% w/w methane.

To improve efficacy, the methanogenesis may be carried out under mixing. This can be achieved using any suitable method or technique including, but not limited to, mechanical mixing (e.g., propellers, pumps) and any other suitable form of mixing.

It is within the skills of those in the art to find suitable methanogenesis conditions and parameters, including materials, temperature, etc. to produce optimal amounts of methane from the hydrolyzed material.

In embodiments, the digestate obtained from methanogenesis is recirculated to the hydrolysis (102, 112, 122).

### Continuous and Semi-Continuous Modes

The present invention is directed to process operating in both, “continuous” and “semi-continuous” modes of operation. According to such operation modes, organic material is fed, decanted and/or removed from the systems and/or processes on a regular basis to achieve a desired digestion target and/or a desired production of biogas.

In embodiments, the process further comprises at least one of: (i) controlling a loading rate of organic material at the hydrolysis step; (ii) controlling a loading rate of slurry hydrolyzed material at the stripping step; (iii) controlling a content of ammonia-stripped slurry and/or amount of digestate at the methanogenesis step.

In embodiments, operating a continuous or semi-continuous mode implies: (i) feeding organic material to the first-stage anaerobic digestion unit one or more times per day; (ii) drawing hydrolyzed material from the first-stage anaerobic digestion unit one or more times per day; (iii) drawing some of the ammonia-stripped slurry one or more times per day; and (iv) drawing a digestate from the second-stage anaerobic digestion unit one or more times per day. In particular embodiments the hydrolysis reactor is fed one or more times per day, and hydrolysed materials are drawn from the reactor and sent for ammonia-stripping once per day.

In embodiments the ammonia-stripping step is operated in semi-batch mode where all influent exits the stripping device after the stripping duration is completed. The ammonia-stripped slurry exiting the stripping device is then fed to the methanogenesis in one or more occurrences per day. Similarly, effluents produced at the methanogenesis step are drawn in one or more occurrences per day.

In embodiments, the continuous or semi-continuous mode comprises controlling an organic loading rate in the first and/or second digestion unit. This can be done for instance by having a holding tank prior to the first digestion unit with valves to control the flow. In the event that biogas production drops, organic loading rate is reduced to reduce any stress on the reactor.

In embodiments, the continuous or semi-continuous mode comprises maintaining a constant temperature in one or more of the first-stage anaerobic digestion units, the NH₃-stripping unit (i.e., ammonia-stripping unit) and second-stage anaerobic digestion unit. In one embodiment, the temperature of the ammonia stripping is maintained constant at about 55° C.

The methods and systems discussed herein provide, among other things, the advantageous feature of being operable in continuous and semi-continuous modes. Indeed, studies prior to the present invention that discussed post-hydrolysis ammonia stripping were limited to batch-mode testing, i.e., a mode wherein all the content of the reactors are fed at one occurrence and drawn after the hydraulic retention duration.

Those skilled in the are aware that testing of batch-mode reactors can identify certain challenges or determine the efficiency of some units, but it is not sufficient to identify challenges in full-scale continuous or semi-continuous mode, which is the most common type of reactors. On the other hand, in continuous or semi-continuous mode like the present invention, accumulation of ammonia and other chemicals can occur in more severity than the batch mode because of continuously feeding and decanting the reactors. Moreover, the testing of continuous or semi-continuous mode is often conducted on larger volumes than batch mode, allowing for better representation of the feedstock and more concrete evidence on the feasibility of post-hydrolysis ammonia stripping. Accordingly, testing continuous or semi-continuous systems as described herein for the first time for a post-hydrolysis ammonia stripping (PHAS) system, advantageously allow identifying operational issues such as accumulation of chemicals.

### B) System for Continuous or Semi-Continuous Two-Stage Anaerobic Digestion

An exemplary system 200 for implementing a process according to the principles described above will now be presented. For example, system 200 may be used to implement the process 100 as described.

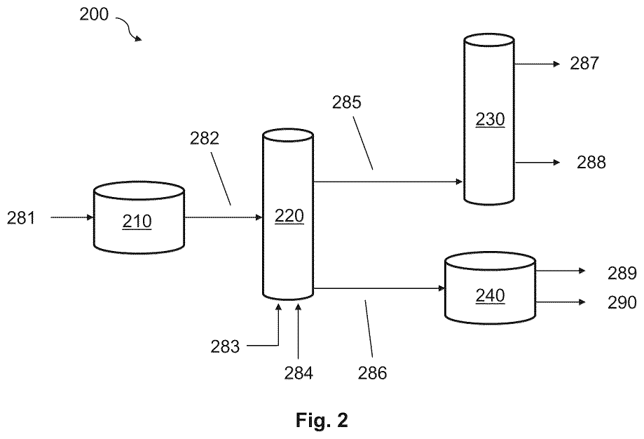

FIG. 2 depicts an example of a system 200 for continuous or semi-continuous two-stage anaerobic digestion. Briefly, in the illustrated embodiment, the system 200 comprises a first-stage anaerobic digestion unit 210, a NH₃-stripping unit 220 (i.e., ammonia-stripping unit) and second-stage anaerobic digestion unit 240. The system may also comprise a NH₃-scrubbing unit 230.

The system operates in a continuous or semi-continuous mode since all the involved units (i.e., first-stage anaerobic digestion unit 210, NH₃-stripping unit 220, and second-stage anaerobic digestion unit 240) may be fed and drawn at any given point in time (continuous mode) or intermittently for one or more times per day (semi-continuous mode).

### First-Stage Anaerobic Digestion Unit 210

The first-stage anaerobic digestion unit is configured to hydrolyze the organic material under anaerobic conditions and to obtain hydrolyzed material which is next fed to the NH₃-stripping unit 220. In the embodiment of FIG. 2, the first-stage anaerobic digestion unit 210 comprises a closed container defining an empty interior for receiving the organic material to be hydrolysed. It also comprises an inlet by which a raw feedstock 281 of organic material is fed into the digestion unit. It further comprises an outlet by which hydrolyzed material 282 is next fed into the ammonia stripping unit.

In embodiments, the digestion unit 210 consists of a tank adapted for hydrolysis of organic materials and content mixing. The tank can be constructed from any suitable material, including concrete and stainless steel, etc. Other construction materials such as wood or steel are possible, but issues of gas leakage and material corrosion may occur. In one embodiment the tank in made of concrete and it comprises an interior lining for ensuring gas-tight performance.

Size of the first-stage anaerobic digestion unit 210 may be adapted according to various parameters such as type of the feedstock 281, volume of feedstock 281 to be treated, amount of water in the feedstock 281, desired flow rate, etc. In one embodiment the size of the first-stage anaerobic digestion unit 210 is determined by multiplying the organic materials daily flow rate by the retention time (in days).

The first-stage anaerobic digestion unit 210 can have one or more mixers to ensure materials are properly mixed. Suitable mixers may include, but are not limited to, mechanical mixers (e.g., propellers, impellers, pumps) and other suitable mixing means.

The first-stage anaerobic digestion unit 210 can also be coupled with or comprise a heating system for providing a temperature-controlled hydrolysis environment (e.g., about 40 to about 45° C., for about five to ten days). In one embodiment the first-stage anaerobic digestion unit 210 is heated through water recirculation in lined water tubes. Preferably the water is heated through sustainable energy such as solar or wind energy, or from an excess energy deriving from the biogas plant.

### NH₃-Stripping Unit 220

The NH₃-stripping unit 220 (or ammonium stripping unit) is configured to strip ammonia from the hydrolyzed material 282 and to eventually obtain an ammonia-stripped slurry 286 which is next fed to the second-stage anaerobic digestion unit 240. Accordingly, the ammonia stripping unit 220 comprises an inlet which is in fluid communication with the outlet of the first-stage anaerobic digestion unit 210 and an outlet by which an ammonia-stripped slurry 286 is next fed to the second-stage anaerobic digestion unit 240.

The ammonia stripping unit 220 can be made from any suitable material, including concrete, steel, stainless steel, etc. that can sustain alkaline stripping pH requirements (e.g., pH of about 9 to about 10). Anti-corrosive materials may be applied to accommodate the stripping pH requirements.

In preferred embodiments, the ammonia stripping unit 220 provides for a gas-to-liquid transfer process wherein a gas carries volatile ammonia as the gas travels through the hydrolyzed material.

The NH₃-stripping unit 220 further comprises an inlet for receiving a gas 283 that is required for the stripping process. Any suitable gas 283 may be used for the stripping process. Depending on desired utilization, the gas 283 may simply be air provided by the surrounding environment. The gas 283 may be a biogas (e.g., regular or upgraded biogas), a RNG, or a mixture thereof. The gas 283 may be stored in tanks under a compressed form prior to be fed into the stripping unit 220. The gas 283 may be a biogas obtained from second-stage anaerobic digestion unit 240 subsequently to the methanogenesis of the ammonia-stripped slurry 286.

In preferred embodiments, increasing pH inside the ammonia stripping unit 220 is favorable to promote ammonia volatility. In embodiments the pH of the stripping is carried out at a pH of about 9.5 to about 10.0, preferably about pH 9.5. Accordingly, the ammonia stripping unit 220 may comprise a separate inlet for receiving an alkaline inflow 284. In one embodiment the alkaline flow 284 is added to the hydrolyzed material inflow 282 in a process called “in-line injection” wherein the alkaline 284 is added into the pipe transporting hydrolyzed material 282 into the ammonia stripping column 220. Alkaline materials may be used as solids or as slurry. The alkaline inflow 284 may comprise any suitable alkaline compound including, but not limited to, lime, NaOH, etc.

The size of the ammonia stripping unit 220 may be adapted according to various parameters such as type of the feedstock 281, flow rate and/or volume of hydrolyzed material 282, desired stripping duration, etc.

In embodiments, the ammonia stripping unit 220 comprises an elongated column configured for gas-to-liquid transfer, the column comprising a higher height-to-width or height-to-diameter ratio. In one embodiment the column is a packed column comprising packed material in the middle of the column, where the hydrolyzed material inflow 282 is sprinkled from the top of the column and a carrier gas 283 (e.g., air, upgraded biogas, biogas) is inserted from the bottom of the column. A solid-liquid separation unit (not shown) may be further provided to reduce clogging inside the column.

In another embodiment, the ammonia stripping unit 220 comprises a bubble column. In this embodiment, the hydrolyzed material 282 is added into the bubble column, and has direct contact with carrier gas 283 at the bottom of the bubble column.

In embodiments, the ammonia stripping unit 220 comprises carrier gas nozzles at the bottom of the unit in a uniform distribution to provide vigorous mixing and well-distributed gas-to-liquid interaction.

In addition, the ammonia stripping unit 220 may be coupled with, or it may comprise, a heating system for providing a temperature-controlled stripping environment (e.g., about 40 to about 55° C. for a few hours or even for days). In one embodiment, the ammonia stripping unit 220 is heated through water recirculation in lined water tubes around its interior (e.g., around the interiors of the stripping column). The water may be heated through sustainable energy such as solar or wind energy, or from an excess energy deriving from the biogas plant.

In embodiments, the ammonia stripping unit 220 is configured to obtain an ammonia-stripped slurry 286 in which ammonia nitrogen has been reduced by at least 50%, or at least 60%, or at least 70%, or at least 75%, or at least 80%, compared to content of ammonia nitrogen present in the hydrolyzed material 282 fed to the ammonia stripping unit 220. For instance, hydrolyzed material 282 leaving the first-stage anaerobic digestion 220 unit may comprise an ammonia nitrogen content of about 2500 to about 6000 mg NH₃—N/L. Accordingly, with a 75% efficiency the ammonia-stripped slurry 286 would comprise about 625 to about 1500 mg NH₃—N/L, and with a 80% efficiency the ammonia-stripped slurry 286 would comprise about 500 to about 1200 mg NH₃—N/L. Preferably, the stripping is such that the ammonia-stripped slurry 286 comprises less than about 3000 mg NH₃—N/L, or less than about 2750 mg NH₃—N/L, or less than about 2500 mg NH₃—N/L, or less than about 2250 mg NH₃—N/L, or less than about 2000 mg NH₃—N/L.

It is within the skills of those in the art to find suitable stripping conditions and parameters, including materials, temperature, etc. to produce an ammonia-stripped slurry 286 suitable for the next steps of the process (e.g., methanogenesis).

### NH₃-Scrubbing Unit 230

In the embodiment shown in FIG. 2, the ammonia stripping unit 220 is also in fluid communication with a NH₃-scrubbing unit 230 such that the NH₃-scrubbing unit 230 can receive, from the ammonia stripping unit 220, a gaseous effluent 285 having a high ammonia content. The NH₃-scrubbing unit 230 is preferably configured for reacting ammonia with sulfuric acid to produce ammonium-sulfate 288 and a gaseous effluent low in ammonia 287 (i.e., a low-NH₃ effluent).

In embodiments, the NH₃-scrubbing unit 230 is configured such that the gaseous effluent low in ammonia 287 is flared or simply discharged to atmosphere. In embodiments, the NH₃-scrubbing unit 230 is configured for recirculating the effluent low in ammonia 287 exiting the NH₃-scrubbing unit 230 into the ammonia stripping unit 220.

Preferably, the NH₃-scrubbing unit 230 is also configured for proper disposal of the ammonium-sulfate 288 produced into the NH₃-scrubbing unit 230 (e.g., discarded, further processed and/or be used as a high-quality fertilizer).

### Second-Stage Anaerobic Digestion Unit 240

The second-stage anaerobic digestion unit 240 is configured for methanogenesis of the ammonia-stripped slurry 286 received from the NH₃-stripping unit 220, and ultimately obtain a digestate 290 and a biogas 289, which may comprise methane and carbon dioxide. As such the second-stage anaerobic digestion unit 240 may comprise a closed container defining an empty interior for receiving the ammonia-stripped slurry 286. It also comprises an inlet which is in fluid communication with the outlet of the NH₃-stripping unit 220. It further comprises an outlet by which digestate 290 may exit the unit and an outlet by which any biogas 289 produced may be collected.

In embodiments, the second-stage anaerobic digestion unit 240 consists of a closed tank adapted for anaerobic digestion and content mixing. The tank can be made from any suitable material, including concrete, steel, stainless steel, wood, etc. that can sustain the requirements associated with anaerobic digestion (e.g., temperature, pH, pressure, etc.). It may contain an interior that comprises a lining or an interior coated with chemical reagents for preventing leakage, rusting and/or corrosion. The size of the tank may be selected according to various parameters including, but not limited to, desired hydraulic retention time and daily flow rate from the ammonia-stripping unit 220.

In embodiments, the second-stage anaerobic digestion unit 240 operates at a constant interior temperature of about 37° C. to about 39° C., and with a hydraulic retention time of about 25 to about 30 days. Accordingly, the second-stage anaerobic digestion unit 240 can also be coupled with or comprise a heating system for providing a temperature-controlled environment. In embodiments the second-stage anaerobic digestion unit 240 is configured such that the interior temperature is maintained using hot water recirculation. In one particular embodiment the second-stage anaerobic digestion unit 240 is heated through water recirculation in lined water tubes. In embodiments, water is heated on-site using renewable and sustainable energy (e.g., solar or wind) or heated using excess energy of the biogas system.

Preferably, the second-stage anaerobic digestion unit 240 is configured for operation under conditions favorable to methanogenic archaea. As such, the second-stage anaerobic digestion unit 240 may further comprise sensors to monitor pH and/or ammonia levels inside the container, as well as controllers for adjusting these and other parameters whenever necessary (e.g., controlling the properties and or amount of the ammonia-stripped slurry 286 fed to the second-stage anaerobic digestion unit 240).

To improve efficacy, the second-stage anaerobic digestion unit 240 may be provided with one or more mixers for mixing contents therein. Suitable mixers may include, but are not limited to, mechanical mixers (e.g., propellers, impellers, pumps) and other suitable mixing means.

Preferably, the second-stage anaerobic digestion unit 240 is provided with or operatively coupled to, a biogas collector (not shown). In one embodiment, the biogas collector comprises a flexible dome positioned on top of the container or tank, the collector being configured to collect and store biogas 289 produced inside the container or tank. The biogas collector may be configured for further purification, compression for storage and/or compression for injection to natural gas pipeline of the biogas 289. The biogas collector may also be configured such that collected biogas 289, upgraded biogas, purified biogas, and/or RNG produced in the second-stage anaerobic digestion unit 240 be redirected into the system 200 and used for ammonia stripping.

Preferably also, the outlet of second-stage anaerobic digestion unit 240 is coupled to a solid-liquid separation device (not shown) of the system 200. Advantageously, separated solids may be used as soil conditioners, whereas liquids can be used for irrigation or recirculated in the system 200 for dilution purposes (e.g., mixed with dry waste before it enters the first-stage anaerobic digestion unit 210).

### C) Using Upgraded Biogas as Stripping Gas

Another aspect of the present invention concerns using upgraded biogas (including but not limited to renewable natural gas (RNG)) for stripping ammonium from hydrolyzed material. According to that aspect, using upgraded biogas for ammonia stripping, instead of air or standard biogas provides unexpected benefits to both post-hydrolysis stripping (i.e., two-stage stripping) and regular side-stripping.

### Two-Stage Stripping Using Upgraded Biogas as Stripping Gas

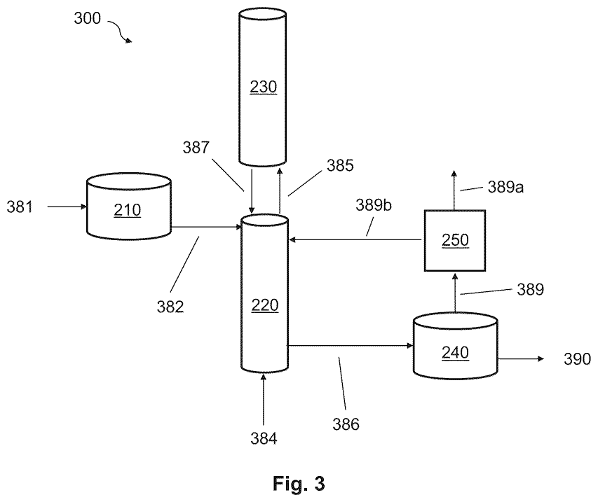

FIG. 3 depicts an example of a two-stage stripping system 300 using upgraded biogas as stripping gas. For example, system 300 may be used to implement process 110 described above. Similar numerals and similar ranges are used to denote similar components as in system 200. Some feeds or outflows present in system 200 may not be described in detail for system 300, and are to be understood to be present but omitted for brevity unless otherwise indicated.

Briefly, like for FIG. 2, the system 300 comprises a first-stage anaerobic digestion unit 210 receiving organic material 381 and outputting hydrolyzed organic material 382 to a NH₃-stripping unit 220 (i.e. ammonia-stripping unit) with an inlet for receiving an alkaline inflow 384, and second-stage anaerobic digestion unit 240 receiving ammonia-stripped slurry 386 and outputting biogas 389 and digestate 390. The system 300 may also comprise a NH₃-scrubbing unit 230 receiving and ammonia-rich outflow 385 and outputting an effluent low in ammonia 387. A biogas collector 250 is provided to upgrade the biogas 389 produced in second-stage anaerobic digestion unit 240 and thereby obtain an upgraded biogas 389a, 389b (e.g., renewable natural gas (RNG)). The upgraded biogas 389b, can then be directed to the NH₃-stripping unit 220 and be used as the stripping gas.

Accordingly, exemplary system 300 differs from system 200 at least in that biogas 389 produced in the second-stage anaerobic digestion unit 240 undergoes collection and processing in biogas collector 250 to obtain upgraded biogas 389b which is then fed to the ammonia stripping unit 220. It will also be understood that the ammonia-scrubbing unit 230 may return the effluent low in ammonia 387 to ammonia-stripping unit 220 to facilitate or improve its operation. It will also be understood that the system 300 may not comprise an inlet for stripping gas 283 as is present in system 200, or the inlet for stripping gas 283 of system 200 may be used to deliver the upgraded biogas 289a.

### Regular Side-Stripping Using Upgraded Biogas as Stripping Gas

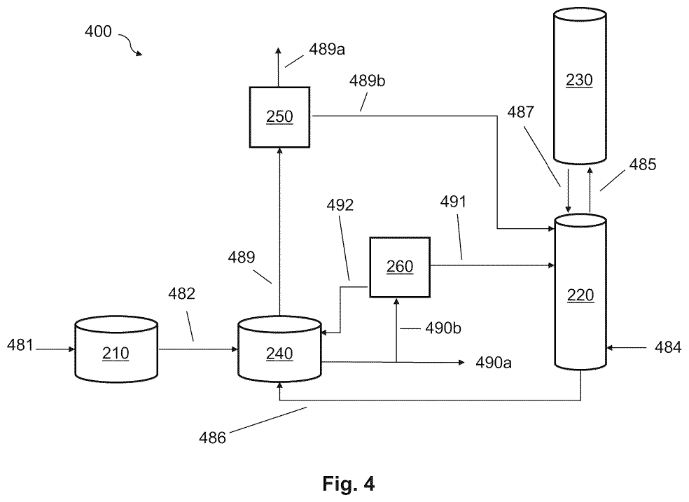

FIG. 4 depicts an example of a system 400 for regular side-stripping using upgraded biogas as stripping gas. System 400 may be used to implement process 120 described above, or other processes according to the principles of the present invention. Similar numerals and similar ranges have been used for system 400 as for systems 200 and 300, and will not be repeated for brevity.

Briefly, the system 400 comprises a first-stage anaerobic digestion unit 210 used as a hydrolyzer to receive raw organic material 481, and a second-stage anaerobic digestion unit 240 used as a methanogenesis stage to convert hydrolyzed organic materials 482 and ammonia-stripped slurry 486 to biogas 489 and digestate 490a, 490b. The system also comprises a NH₃-stripping unit 220 (i.e. ammonia-stripping unit) with an inlet for receiving an alkaline inflow 484, a NH₃-scrubbing unit 230 for receiving an ammonia-rich effluent 485 and outputting an effluent low in ammonia 487, and an optional solid/liquid separation unit 260. A biogas collector 250 is further provided to upgrade biogas 489 produced in second-stage anaerobic digestion unit 240 and thereby obtain upgraded biogas 489a, 489b (e.g., renewable natural gas (RNG)).

Therefore, unlike FIGS. 2 and 3 which illustrate post-hydrolysis ammonia stripping, in the system 400 illustrated at FIG. 4, the NH₃-stripping unit 220 is located after a second-stage anaerobic digestion unit 240 and it thus treats a certain percentage of a working volume (1-20%) of digested 490b from the second-stage anaerobic digestion unit 240.

Indeed, the second-stage anaerobic digestion unit 240 produces a digestate outflow (490a, 490b). A portion of the digestate outflow 490a may be collected for further processing, such as for biosolids processing. Another portion of the digestate outflow 490b may be processed to obtain and ammonia-stripped slurry 486 which is returned into second-stage anaerobic digestion unit 240. For instance, digestate stream 490b may be fed into a solid/liquid separation unit 260 to obtain a liquid portion 491 and a solid portion 492. The liquid portion 491 may be further fed to the ammonia stripping unit 220, while the solid portion 492 may be returned to the second-stage anaerobic digestion unit 240.

It will be understood that the solid/liquid separation unit 260 may operate at a variety of efficiencies, flowrates and retention times. Suitable adaptations to the solid/liquid separation unit 260 and to the ammonia-stripping column will be apparent to skilled persons and are to be considered within the scope of the present disclosure.

In the illustrated embodiment, at least a portion of the upgraded biogas 489b obtained, from the biogas collector 250 is directed to the NH₃-stripping unit 220 and is used as stripping gas. Using upgraded biogas such as RNG as a stripping gas means that NH₃-stripping may be carried out at similar conditions as described herein above, except that the NH₃-stripping 220 is for treating the digestate from the second-stage anaerobic digestion unit 240 instead of treating hydrolyzed material from the first-stage anaerobic digestion unit 210.

Benefits and Conditions Associated with the Use of Upgraded Biogas as Stripping Gas

In accordance with the present invention, using upgraded biogas for ammonia stripping, instead or air and/or standard biogas, provides unexpected benefits to both, post-hydrolysis stripping (i.e., two-stage stripping) and regular side-stripping.

In accordance with the present invention, efficiency of the ammonia removal processes is improved when the pH of the hydrolyzed material requiring ammonia stripping is high (e.g., pH of 9 or higher). Typically, biogas comprises 40-60% CO₂. However, CO₂ reacts with hydrolyzed material and forms carbonic acid, which lowers the pH and hence lowers the ammonia removal efficiency rapidly. Therefore, using upgraded biogas (i.e. depleted from CO₂) ensures a better efficiency of the NH₃-stripping unit compared to regular biogas by preventing the formation of carbonic acid and related pH decrease.

Another advantage of using upgraded biogas is related to the fact that existing systems that use regular biogas for stripping usually must conduct a continuous ammonia stripping because the flowrate of biogas has to be minimal to reduce its impact on pH (i.e., acidification). This means that the ammonia stripping unit must be heated at all times and extremely high pH levels (pH>10) are required. Using upgraded biogas in accordance with the present invention in a semi-batch mode may allow to reduce the operation of the ammonia stripping unit to only a few hours per day (e.g., 2-3 hours per day), thereby substantially reducing the energy needed for heating the ammonia stripping unit as well as reducing the alkaline requirements to raise the pH.

Using upgraded biogas may also provide benefits compared to ambient air. Although air comprises only about 0.03% CO₂, air also comprises about 21% oxygen. Since upgraded biogas is oxygen-free and it can thus be more favorable because oxygen can negatively affect the process. Therefore, one of the main advantages or benefits of using upgraded biogas over air is that upgraded biogas increases the anaerobic nature of the process. In embodiments, the stripping with upgraded biogas thus excludes stripping with air.

Using upgraded biogas for the stripping and/or stripping under anaerobic conditions may also prove to be favorable to methanogenic archaea and/or avoid reducing abundance of Firmicutes. Indeed, air can be toxic for some microorganisms and using upgraded biogas can therefore help maintain the microorganism activity.

Nevertheless, those skilled in the art understand that, compared to air, upgraded biogas must be dealt with more carefully for safety reasons. Therefore, in accordance with the present invention, in embodiments the system is configured such that pipelines, valves, units, etc. are compatible with upgraded biogas and are explosion proof whenever applicable.

In embodiments, the stripping with the upgraded biogas is carried out at a constant temperature of about 40° C. to about 65° C., or about 55° C. In embodiments, the stripping with the upgraded biogas is carried out at a pH of about 9 to about 10, or about 9.5.

In embodiments, stripping with upgraded biogas removes at least 20%, or at least 30%, or at least 40%, or at least 43%, or at least 45%, at least 50%, or at least 55%, or at least 60%, or at least 62%, or more ammonia than stripping with biogas (i.e., not upgraded biogas).

In embodiments, stripping with upgraded biogas reduces energy consumption, material consumption and/or spatial requirements for the systems 300 and 400 by at least 5%, or at least by 10%, or at least by 20%, or at least by 30%, or at least by 40% or at least by 50%, compared to stripping with biogas (i.e., not upgraded biogas).

Those skilled in the art will recognize, or be able to ascertain, using no more than routine experimentation, numerous equivalents to the specific procedures, embodiments, claims, and examples described herein. Such equivalents are considered to be within the scope of this invention and covered by the claims appended hereto. The invention is further illustrated by the following examples, which should not be construed as further or specifically limiting.

### EXAMPLES

### Example 1: Post-Hydrolysis Ammonia Stripping Enhances Two-Stage Anaerobic Digestion of Poultry Manure

Post-hydrolysis ammonia stripping was investigated as a new approach to enhance the methane potential of high ammonia substrates, such as poultry manure. The objective was to address some of the noticeable disadvantages in the existing ammonia-stripping techniques i.e., treatment of raw samples and side-stream stripping.

This study was conducted as reported in the scientific publication Adghim M. et al., Journal of Environmental Management 319 (2022) 115717, which is incorporated herein in its entirety.

Briefly, ammonia stripping was performed on hydrolyzed samples of poultry manure (PM) alone or mixed with a mixture of substrates (MS) that consisted of coffee ground (23.3% w/w) and cheese factory wastes composed of process water (34% w/w) and sludge (42.6% w/w). PM and MS were mixed at different volatile solids (VS) mass ratios of PM:MS 100:0, 75:25, 50:50, 25:75, 0:100.

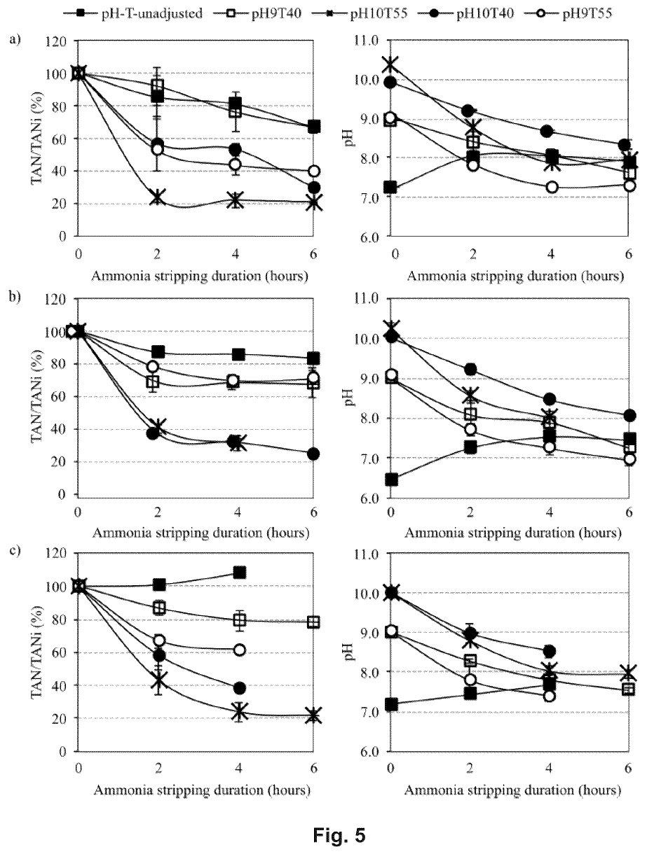

Present FIG. 5 (corresponding to FIG. 2 in the 2022 publication), shows ammonia and PH levels during ammonia stripping of a) PM100, b) PM75:MS25, and c) PM50:MS50. Ammonia levels started at around 4890, 3450, and 3040 mg NH3-N/L in PM100, PM75:MS25, and PM50:MS50, respectively.

As shown in FIG. 5, post-hydrolysis ammonia stripping removed up to 78% of ammonia. All samples with initial pH of 10 showed higher ammonia removal (average of 73%) than those set at pH 9 (average of 36%) or unadjusted (average of 14%). Increasing the pH was more effective than increasing the temperature for removing ammonia as the removal efficiency increased on average by 74, 136, and 143% when pH was increased from 9 to 10 in PM100, PM75:MS25, and PM50:MS50, respectively.

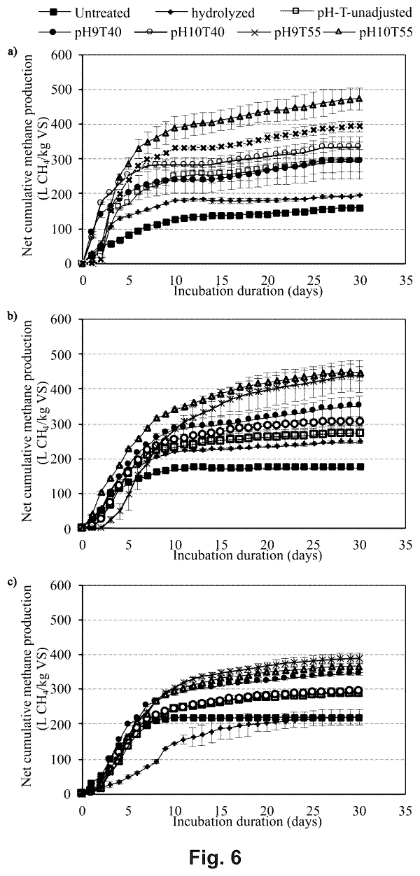

Present FIG. 6 corresponds to a modified version of FIG. 5 in the 2022 publication. This figure shows the net cumulative biomethane potential of the untreated, hydrolyzed, and ammonia-stripped samples. Samples treated at a higher temperature i.e., 55° C., yielded higher methane potential when compared to other samples even though ammonia levels were slightly higher in some cases. Actually, increasing the stripping temperature to 55° C. improved methane potential by 41%.

Samples stripped at pH 10 and temperature of 55° C. had up to 197% and 150% more methane potential when compared with the untreated and hydrolyzed samples, respectively.

### Example 2: Comparative Testing of Air and Renewable Natural Gas as Mediums for Ammonia Stripping to Enhance Biogas Potential

### 1. Summary

The possibility of using renewable natural gas (RNG) for ammonia stripping in anaerobic digestion of poultry manure (PM) applications was investigated and compared with ammonia stripping with air for the first time. RNG and air led to comparable ammonia removal efficiencies of up to 60 and 69% under elevated pH and temperature (9.5 and 55° C.), respectively. The consequential improvement due to these treatments on biogas production was 58% and 70% for samples treated with RNG and air, respectively.

### 2. Methodology

2.1 Sample collection: Poultry manure (PM) samples were collected from layer chickens on an egg farm located in Ottawa, Canada. The manure was scraped from the floors and transported via a conveyor to an onsite pile. Since the farm does not implement any bedding systems, the collected manure had few contaminants which mainly constituted of feathers. PM samples were characterized shortly after collection and stored at 4° C. throughout the experiment. PM had 30.0±0.3% total solids (TS) and 22.9±0.2%. The sample had high total ammonia and (TAN) total Kjeldahl nitrogen (TKN) values of 2496±74 mg/L and 12976±381 mg/L, respectively. The high organic nitrogen content, which was about 80.8% of TKN, indicates that ammonia fermentation could lead to extremely high ammonia levels causing inhibition of microorganism activities. PM samples were diluted to 12% using distilled water to facilitate the hydrolysis and ammonia stripping of the samples. This had led to reducing nitrogen levels by a factor of 2.5, which was still not enough for ammonia levels to be below previously reported inhibitory levels (above 2000-2500 mg NH₃—N/L) (Y. Chen et al., 2008; Usack & Angenent, 2015). The inoculum (I), on the other hand, was collected from a mesophilic digester that operates on cow manure and corn silage near Ottawa, Canada. Its TS %, VS %, TAN, and TKN are 4.9±0.1% and 3.8±0.1%, 1592±5 mg/L, and 3211±157 mg/L, respectively. The inoculum was characterized shortly after collection and stored at 35-40° C. before being used. Table 1 shows the rest of the PM and I's characteristics.

TABLE 1
Poultry manure and inoculum characteristics

| Characteristic | Unit | PM | I |
|---|---|---|---|
| pH | | 8.63 | 7.61 |
| TS | % | 30.0 ± 0.4 | 4.9 ± 0.1 |
| VS | % | 22.9 ± 0.2 | 3.8 ± 0.1 |
| VS/TS | % | 76% | 78% |
| VFA | g CH₃COOH/L | 14.2 ± 0.8 | 7.3 ± 0.4 |
| COD | g COD/L | 84.3 ± 9.2 | 48.5 ± 0.5 |
| ALK | g CaCO₃/L | 25.7 ± 0.2 | 10.3 ± 0.05 |
| TAN | g NH₃—N/L | 2.4 ± 0.07 | 1.5 ± 0.01 |
| TN | g N/L | 13.2 ± 0.5 | 3.2 ± 0.4 |
| NO₃—NO₂ | g N/L | 0.2 ± 0.02 | 0.2 ± 0.01 |
| TKN | g N/L | 12.9 ± 0.5 | 3.2 ± 0.2 |

2.2 Hydrolysis: PM was diluted to 12% TS and blended in a food blender for only 10-15 seconds to minimize the impact of heating due to blending on hydrolysis. Large contaminants like feathers were removed by sieving the blended PM through a ⅛″ mesh. The sieved PM was then filled into four 500 ml bottles, leaving 10-15% headspace. No chemicals were added to ensure that the hydrolysis is only occurring due to biological activities. The bottles were then purged with nitrogen to provide anaerobic conditions for the hydrolysis. The bottles were placed in a shaking incubator where the temperature and the shaking speed were set to 40° C. and 150 rpm, respectively. Samples were collected every day for pH, TAN, TKN, and VFAs analyses. The main purpose of the hydrolysis and acidogenesis stage in this study was to biologically promote organic nitrogen conversion to ammonia, hence, the hydrolysis setup resumed until TAN levels stabilized (around 5 days), after which the effluents were fully characterized.

2.3 Ammonia stripping: Ammonia stripping was conducted in 150 ml cylinders containing 120 ml of hydrolyzed PM. The samples were pre-heated to the stripping temperature (40° C. or 55° C.) using a water bath, and then the pH was adjusted to 9.5 using lime (Ca(OH)₂); around 24 g lime/kg PM was needed. Air and renewable natural gas (RNG) were used separately for stripping and comparison purposes. RNG consisted mainly of methane (94% v/v) and ethane (5% v/v). The flow rate was set to 100 L gas/L sample/hour and the tube was placed at the bottom of the beaker with a diffuser at its end to supply finer bubbles. The samples were labelled as T (stripping temperature)-gas medium i.e., T55-Air, T55-RNG, T40-Air, and T40-RNG. The stripping continued for three hours. Samples were taken every half an hour to measure ammonia and pH, however, the samples were fully characterized after the stripping duration was over. Each scenario was tested using triplicates.

2.4 Batch BMP test: A batch biochemical methane potential test (BMP) was set up for the raw, blended, hydrolyzed, and ammonia-stripped samples at a mesophilic temperature (35-40° C.). Samples were inoculated at an inoculum to substrate ratio (ISR) of 1-2. The inoculum was degassed for a few days before being used in the BMP test and then used as a blank to subtract the contribution of the inoculum to biogas production and allow the reporting of net biogas production of the PM samples. Samples and inoculums were added to 250 ml BMP bottles where 30% headspace was maintained, and the samples were purged with nitrogen to ensure anaerobic conditions in the bottles. BMP bottles were then placed in a shaking incubator at 35° C. and 150 rpm. Biogas production was measured daily using water displacement, and biogas characterization of methane content was conducted once a week. The BMP test resumed until the biogas production rate was less than 1% of the cumulative biogas production for at least three consecutive days. The digestate was fully characterized at the end of the BMP test.

2.5 Analytical methods: Samples were analyzed shortly after collection at any stage to ensure accurate results. TS and VS were determined using standard method no. 2540 from APHA. VFAs were measured using the Esterification method: Hach TNT872 (50-2500 mg CH₃COOH/L); COD was measured using the Reactor Digestion Method: Hach TNT822 (20-1500 mg COD/L); total alkalinity was measured using colorimetric method 10239: Hach TNT870 (25-400 mg CaCO₃/L); TAN was measured by Salicylate method: Hach TNT (2-47, 100-1800 mg NH₃—N/L). Methane content in the biogas was measured using a 5′×0.125″ SS 100/120 HayeSep™ T column fitted in a gas chromatography instrument (GOW-MAC™ Series 400, Bethlehem, PA). Helium was used as a carrier gas, the temperatures of the column, detector, and injector were set to 100, 100, and 120° C., respectively, and the current was set to 100 mA.

### 3. Results and Discussion

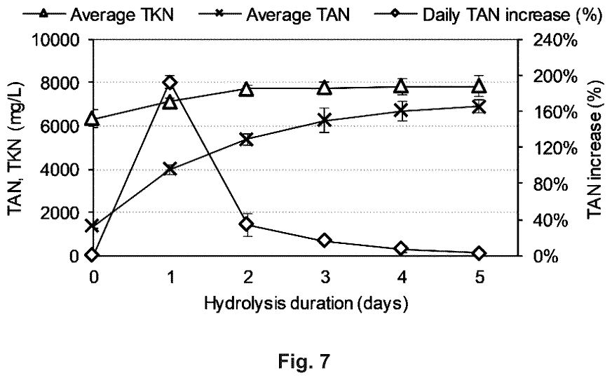

3.1 Ammonia fermentation and stripping: The incubation of poultry manure for five days increased TAN levels from around 1367 mg NH₃—N/L to 6895 mg NH₃—N/L, translating to around a 400% increase in ammonia levels. The highest rate of organic nitrogen conversion to ammonia occurred on the first day of incubation (around 192% increase in ammonia levels) and then sharply declined to 34% on the second day. The slight increase in TKN levels shown in FIG. 7 indicates that moisture has been lost during hydrolysis, however, the post hydrolysis analysis showed that TS % reduced from 12% to 8.8% which means that the impact of VS destruction on TS % during the hydrolysis was higher than the impact of evaporation. This observation was also supported by the transformation rates of lipids and fats to volatile fatty acids, which increased from 7600 to 20810 mg CH3COOH/L. At the end of the incubation period, around 88% of organic nitrogen had been converted to ammonia, which is comparable to the 90% conversion rate observed by Sürmeli et al. (2017) for biological hydrolysis of chicken manure. This high conversion rate of organic nitrogen, followed by ammonia stripping, is meant to limit further increases in ammonia levels during methanogenesis.

For T55-Air and T55-RNG, the effluents of the hydrolyzers were heated for 25 minutes in a water bath to increase the temperature from 40° C. to 55° C. prior to pH adjustment. The preheating step was essential to start the stripping at the right pH level i.e. 9.5 as pH drops when the temperature increases (Bonmatí & Flotats, 2003). Heating the samples, as well as the addition of lime for pH adjustment, had increased the TS % to an average of 16.5 and 15.9% in T55-Air and T55-RNG, respectively.

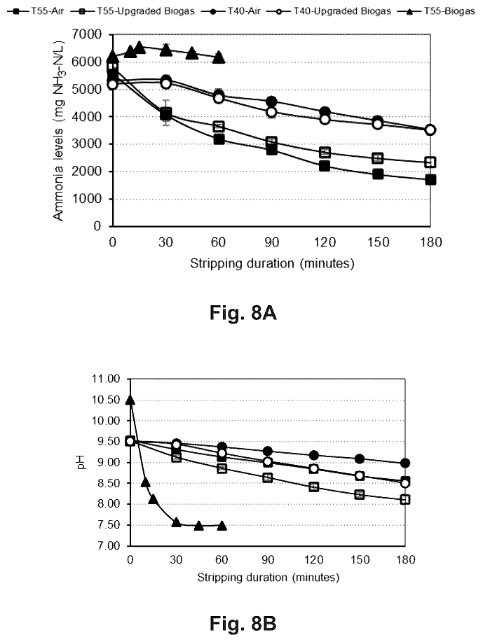

Ammonia levels were successfully reduced in all the tested scenarios (FIG. 8). Samples treated at 55° C. underwent significantly higher ammonia removal efficiencies than those treated at 40° C., despite the gas medium used. This is because FAN levels were higher, and thus more volatile; the initial levels of FAN at pH 9.5 and temperatures of 55° C. and 40° C. averaged 5212 and 4358 mg NH₃—N/L. Most of the removal occurred in the first 90 minutes for all scenarios, after which the ammonia removal rate dropped significantly due to the lower pH, which dropped the volatility of the ammonia.

Air and RNG were both capable of achieving high ammonia removal efficiencies and were comparable at more conservative testing conditions (higher temperature). One of the perceptible differences in air and RNG performances as stripping gases is that RNG had resulted in a slightly higher rate of pH drop. For example, pH dropped to an average of 9.31 and 9.12 in the first 30 minutes of stripping with air and RNG, respectively. Despite this difference in pH levels at that point, both air and RNG had achieved 28% ammonia removal, indicating that this level of pH was still feasible for ammonia removal. Having said that, the effect of pH drop on the ammonia removal efficiency was more obvious towards the end of the stripping duration. The impact of RNG on pH could be due to the higher impurities in the used RNG (5% v/v ethane), which can react with moisture and form ethyl alcohol that can act as a weak acid (W. Chen et al., 2021).

The results of ammonia stripping using RNG are promising and noteworthy. They present several advantages when compared with using biogas as a stripping medium. RNG successfully removed more than 50% of ammonia under gentler conditions than those needed for stripping with biogas. For instance, ammonia removal with biogas requires pH levels that are higher than 10, temperatures higher than 60° C., and prolonged durations, to achieve high ammonia removals (Serna-Maza et al., 2014; Zhang et al., 2017). The primary advantage of RNG to biogas is the low CO₂ content in the former, which avoids abrupt drops in pH and the volatility of ammonia. As RNG in biogas plants can be integrated with conventional natural gas pipelines, the utilization of RNG within the plant for ammonia removal purposes should not pose additional challenges than those that biogas recirculation would. Having said that, provisions of ammonia and moisture traps must be accounted for to recover the RNG's quality before injection into the natural gas grid. Alternatively, instead of injection into the natural gas grid, the RNG used for stripping could be recirculated in a closed loop and replaced or endorsed with new RNG when needed.

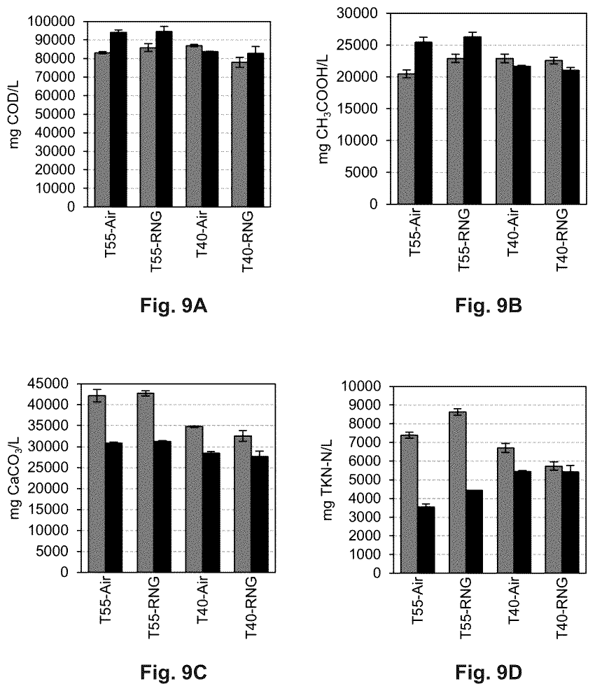

3.2 Impact of ammonia stripping on other characteristics: Effluents of the ammonia stripping setups were fully characterized to have an accurate estimation of what is going into the second stage of AD. FIG. 9 shows the characteristics of the samples immediately before and after stripping for three hours. There were mainly two factors affecting the samples' characteristics during the stripping, which are moisture loss and stripping. TKN decreased in all samples because stripping of ammonia was significant. TKN reduction shows the impact of moisture loss and stripping together i.e., if there is no impact from heating the sample, TKN and TAN reductions would be the same, however, TKN reduction is less due to the loss of moisture. Alkalinity had also decreased in all stripping setups. This could be explained by the limewater and CO₂ reaction which forms a precipitate of CaCO₃ which also affects the ammonia removal efficiency.

On the other hand, VFAs and COD had increased in all ammonia stripping setups except for T40-Air. As mentioned above, while heating worked on increasing the concentration, stripping with air at a lower temperature led to a greater impact of stripping. Subjecting the samples to air for a prolonged period may have enabled some mesophilic aerobic activities that led to the consumption of VFAs and sCOD in T40-Air. Conversely, the impacts of evaporation on COD and VFAs were more prevalent under higher temperatures or whenever RNG was used. It is important to note that since the percentages of COD or VFA removals were not high (<5.5%), there were no apparent adverse effects on the digestibility of the samples treated with air in the methanogenesis stage. The impacts of stripping on the samples' characteristics are not widely discussed in the literature. However, similar results were reported by Fakkaew and Polprasert (2021) in terms of VFA, COD, TAN, and TKN variations due to stripping. Having said that, they have reported a noticeably lower increase in TS when compared to this study (5% versus 60%, respectively). This could be due to the high moisture content in this study compared to the other (85% versus 70%, respectively).

### 3.3 Methane Production

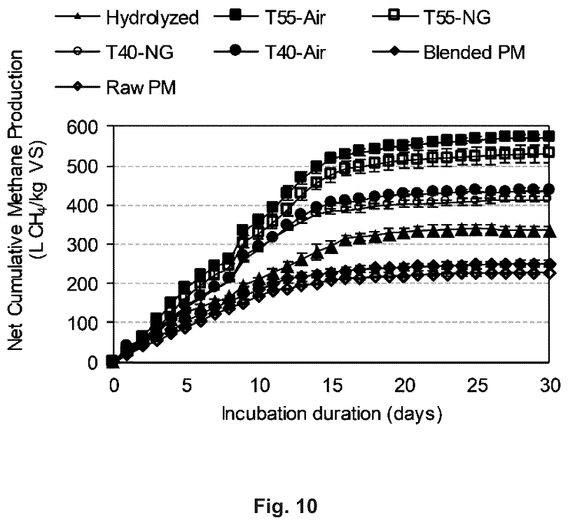

3.3.1 Impact of ammonia stripping: Biogas production was improved in all tested ammonia stripping scenarios (FIG. 10). Hydrolyzed samples that were not subjected to any treatment had encountered inhibition which limited their methane production to 338±12 L CH4/kg VS. Post BMP characterization indicates that the low methane production was due to high ammonia levels of 3050 mg NH₃—N/L. On the other hand, ammonia stripping significantly improved methane production (p=2.4×10⁻⁸) by 71, 58, 30, and 24% in the cases of T55-Air, T55-RNG, T40-Air, and T40-RNG, respectively. This improvement in methane production could be traced to the lower ammonia levels in the digestate, which were 1867, 2021, 2352, and 2402 mg NH₃—N/L for T55-Air, T55-RNG, T40-Air, T40-RNG, respectively.

There is no evidence that stripping with air had any negative impact on methane production or the microbial population. This is because the stripping was done at an intermediate stage where methanogens' presence was already minimal. Despite stripping with air, dissolved oxygen (DO) levels at the end of the stripping did not exceed 0.35 mg/L, which is well below the inhibitory levels of DO (>2 mg/L) reported by (Botheju et al., 2011). Moreover, Fakkaew and Polprasert (2021) reported that DO levels of 2.5 mg/L of stripped digestate did not impact the methanogenic activities. The improvement of methane potential due to air stripping in this study was in line with previously reported literature (Huang et al., 2019; Li et al., 2018). On the other hand, the impact of stripping with RNG on the methane potential is promising and yielded comparable results to stripping with air. Compared with previously reported methane production enhancement using biogas (Nielsen et al., 2013; Serna-Maza et al., 2014), RNG can achieve higher methane production and alleviate ammonia inhibition under significantly less severe operating conditions.

Alongside ammonia reduction, heating the samples at 55° C. for 3-3.5 hours in the T55-Air and T55-RNG stripping setups increased the COD and VFAs levels as indicated in FIG. 9 and therefore increased the digestibility of the sample.

3.3.2 Impact of blending and hydrolysis: To better understand the methane potential of poultry manure without ammonia targeted treatment, three sets of controls were tested in the BMP test (FIG. 10): 1) raw PM, 2) blended PM, and 3) hydrolyzed PM. Blending the sample and sieving it using 0.8 mm mesh slightly improved the methane potential of Raw PM (improvement of 9.8%).

The hydrolyzed sample (representing two-stage AD) had improved the methane potential of the blended sample (representing one-stage AD) by 37.4% despite having almost the same ammonia levels (around 3000 mg NH₃—N/L). The stability and optimal biogas production in two-stage systems were also highlighted in (Nasir et al., 2012; Pan et al., 2013). Since the effluents of both setups showed similar chemical characteristics, the difference in BMP results can be justified by the substantial increase of ammonia levels by 93% during the digestion of blended PM, whereas ammonia levels only increased by 6% during the digestion of hydrolyzed PM. Moreover, the low pH in the separate hydrolysis/acetogenesis stage of PM increased VFAs from around 7667 to 21869 mg CH₃COOH/L by converting short-chain fatty acids and other intermediate products into acetic acid, which is readily digestible by methanogens.

Despite the clear inhibition of biogas production in all PM controls, their performance was unexpectedly close to those of less problematic types of feedstock such as dairy manure, which typically produces between 200-250 L CH₄/kg VS without any treatment (Huang et al., 2016; Usack and Angenent, 2015). However. it should be noted that the results in this study are based on batch BMPs, whereas accumulation of ammonia in continuous or semi-continuous systems running on PM is more likely to occur than in systems running on dairy manure. Therefore, ammonia removal from PM would still be highly recommended for continuous and semi-continuous applications.

4. Conclusion: The removal of ammonia from renewable natural gas or air was tested at multiple conditions and appeared to perform comparably under similar conditions. The use of renewable natural gas for stripping was found to be promising and effective in improving the methane potential of high ammonia feedstock. Therefore, stripping with natural gas can be considered an efficient anaerobic stripping medium that is readily available in biogas plants with biogas upgrading systems.

### Example 3: Semi-Continuous AD Process Coupled with Post-Hydrolysis and Side Stream Ammonia Stripping

### 1. Summary

Poultry manure mono-digestion in semi-continuous mode was experimentally evaluated using two different ammonia stripping configurations aimed at reducing the inhibitory effects of ammonia 1) Post-hydrolysis (PHAS) and 2) Side-stream ammonia stripping (SSAS). Ammonia stripping operating conditions were set to pH 9.5, 55° C., and flowrate of 100 L gas/L/hour. Air and renewable natural gas (RNG) were tested as stripping mediums. PHAS outperformed SSAS in both air and RNG stripping. Volumetric and specific biogas production from PHAS and SSAS scenarios averaged up to 1.91 and 1.26 L/L/day and 831 and 701 L biogas/kg VS·day under organic loading rates of 2.61 and 1.8, respectively.

### 2. Materials and Methods

2.1 Substrates and inoculum: Poultry manure (PM) samples were collected from layer chickens in an egg farm in Ottawa, Canada. The manure is scraped from the floors and transported via a conveyor to an outdoor pile. Since the farm does not implement any bedding systems, the collected manure had few contaminants, mainly comprised of feathers. Around 200 kg of PM was collected and characterized shortly after collection and before storing at −18° C. to limit biodegradation and preserve the sample throughout the experiment. Portions of PM were thawed for one day before being used for the experiment. PM had 24.3±0.1% total solids (TS) and 16.0±0.1% volatile solids (VS). PM had high total ammonia (TAN) and total Kjeldahl nitrogen (TKN) values of 4356±123 mg/L and 9398±106 mg/L, respectively. The high organic nitrogen content, about 53.6% of TKN, indicates that ammonia fermentation could lead to extremely high ammonia levels causing inhibition of microorganism activities (W. Huang et al., 2016). Volatile fatty acids (VFA), chemical oxygen demand (COD), alkalinity, and pH of PM were 28713±459 mg CH₃COOH/L, 170408±7688 mg COD/L, 39762±320 mg CaCO₃/L, and 8.63, respectively. The inoculum was collected from a mesophilic digester that operates on cow manure and corn silage near Ottawa, Canada. Its TS %, VS %, TAN, TKN, VFA, COD, ALK, and pH are 4.9±0.1% and 3.8±0.1%, 1592±5 mg NH₃—N/L, 3211±157 mg NH₃—N/L, 7300±400 mg CH₃COOH/L, 48500±500 mg COD/L, 10300±50 mg CaCO₃/L, and 8.2 respectively. The inoculum was characterized shortly after collection and stored at 35-40° C. before being used for the batch and the beginning of the semi-continuous experiments.

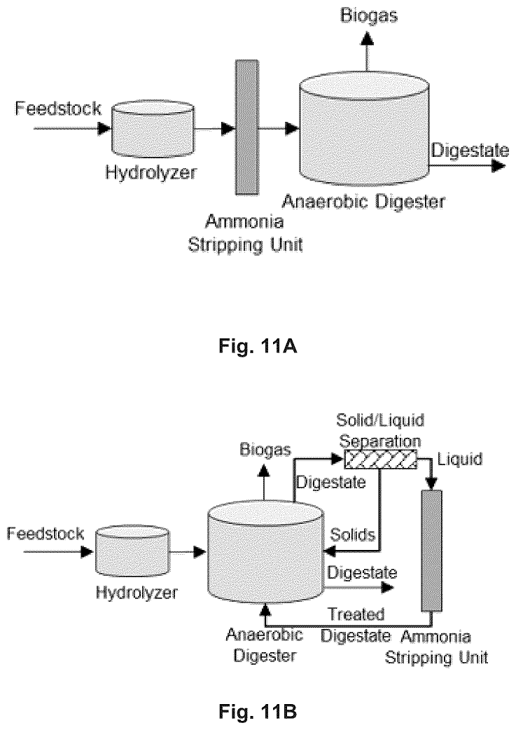

2.2 Experimental setup for semi-continuous two-stage AD systems: The setup for both post-hydrolysis ammonia stripping (PHAS) and side-stream ammonia stripping (SSAS) in a two-stage semi-continuous configuration consisted of three main vessels: 1) hydrolyzers, 2) ammonia-stripping units, and 3) main digesters (methanogenesis tank) in the order presented in FIGS. 11A and 11B. Two systems were operated simultaneously under similar conditions to serve as duplicates.

The overall duration of the experiment was 400 days, divided into different scenarios, as presented in Table 2. The experiment started with filling the main digesters with inoculum up to the working volume (10 L), and the start-up phase included feeding the reactors daily with diluted PM with reduced TAN levels (800 mg NH₃—N/L) to avoid shocking the microorganisms. Then, the feeding was increased gradually every week from 0.5 to 2.6 g kg VS·day, which was the target OLR corresponding to the selected hydraulic retention time (20 days) and working volume (10 L). Then, the reactors were fed with ammonia-stripped hydrolyzed PM in PHAS scenarios, whereas the reactors were fed with hydrolyzed PM (not ammonia-stripped) in SSAS scenarios. The stripping was conducted following the procedures listed hereinbefore. The sequence of the scenarios was determined by anticipating the least to the most impactful scenario in terms of ammonia inhibitory effects. Therefore, the control scenario (no treatment) was conducted at the end of the experiment. Each scenario lasted for at least three hydraulic retention time (HRT) or until the biogas production, as well as the digestate characteristics, were consistent (less than 10% variation per day) for at least one HRT (Usack et al., 2012; Usack & Angenent, 2015).

TABLE 2
Annotation and description of the different
phases in semi-continuous reactor experiments

| | | | Stripping | |
|---|---|---|---|---|
| Annotation | Start | End | medium | Stripped Portion |
| Startup | 0 | 48 | N/A | 0 |
| PHAS-1 | 49 | 126 | Air | 100% of hydrolysis reactor |
| | | | | effluent (500 g/day) |
| PHAS-2 | 127 | 163 | RNG | 100% of hydrolysis reactor |
| | | | | effluent (500 g/day) |
| SSAS-1 | 164 | 224 | RNG | 20% of reactor volume per |
| | | | | day (2 kg per day) |
| SSAS-2 | 225 | 285 | RNG | 10% of reactor volume per |
| | | | | day (1 kg per day) |
| SSAS-3 | 286 | 332 | Air | 10% of reactor volume per |
| | | | | day (1 kg per day) |
| Control | 333 | 400 | N/A | Reactors fed with hydrolyzed |
| | | | | PM (no treatment) |

2.3 Sample preparation: Every five days, a bucket of PM was thawed for one day, then it was used to prepare the feedstock for the following five days. PM was diluted to 10% TS and blended in a food blender for only 10-15 seconds to minimize the impact of heating due to blending on hydrolysis (Holliger et al., 2016). Large contaminants like feathers were removed by sieving the blended PM through a ⅛″ mesh. The sieved PM was then stored in 5 L buckets to feed the hydrolyzers. This process is repeated throughout the experiment.

2.4 Hydrolysis reactors design: The hydrolyzers were designed based on the back-calculations for the organic loading rate (OLR), which was set to be around 2.6 g VS/L·day. The OLR was deliberately set at the lower range reported by literature (2-6 g VS/L·day) (Fernandez-Gonzalez et al., 2019; Nie et al., 2015) to have a better resolution of the impact of treatment. Two cylindrical hydrolysis reactors were manufactured from plexiglass with a 22.9 cm diameter and 15.9 cm height. The reactors' HRT and working volume were set to 5 days and 3 liters, respectively. The hydrolysis HRT was selected based on a previous study (Adghim et al., 2023). The reactors design provisioned sampling ports to incorporate feeding and decanting, as well as a heating rod and thermometer.

The reactors were placed on shakers and set at 50 rpm, the maximum rpm to achieve stable rotation and proper mixing of the reactor's contents. The reactors were heated using a thermocouple rod inserted into the center of the reactor, and the heating was controlled by connecting both the heating rod and the thermometer to a temperature control device, which was programmed to stop heating when the temperature of the material reached 40° C. and restart when the temperature dropped to 35° C. The reactors were fed once a day with 600 mL of diluted PM (10% TS), and the same amount was decanted to maintain the working volume.

The primary purpose of the hydrolysis step in this experiment was to ferment organic nitrogen and convert it to ammonia through biological pathways. Therefore, no acid or alkali was added to enhance or expedite hydrolysis. Furthermore, the temperature of the reactor was set to 40° C., which does not induce the thermal breakdown of organic compounds but provides hydrolytic enzymes and acidogenic microorganisms a suitable environment to utilize the organic compounds and transform them into hydrolysis products (Fisher et al., 2019; Romero-Güiza et al., 2014).

2.5 Main digesters design: Two duplicate reactors were used as methanogenesis vessels (AD1 and AD2) throughout this experiment. The design criteria were similar to the hydrolysis reactors, except for HRT, which was set to 20 days. This HRT was determined based on a previous biochemical methane potential (BMP) test conducted in a previous study (Adghim et al., 2023). The diameter and height of the reactors were 30.5 and 27.9 cm, respectively. The reactors were heated using a similar configuration to the hydrolysis reactors. The reactors were fixated on shakers at the speed of 35 rpm, which was the maximum achievable speed to ensure proper mixing and stability of the reactor. The larger volume of the main digesters compared to the hydrolyzers required less rotational speed to achieve proper reactor stability. The proper mixing was evaluated visually and by weekly monitoring of changes in total solids at the bottom of the reactors to detect sedimentation. The biogas was collected in 40 L gas-impermeable bags, which were emptied every day to measure the volume of the produced biogas. Biogas production was adjusted to standard temperature and pressure at 0° C. and 1 atm. Methane content in the biogas was measured using gas chromatography (GC). The pH of the digestate was recorded daily and immediately after decanting to avoid changes in pH due to CO₂ desorption and drop in temperature, which could lead to an increase in pH levels and erroneous representation of the digestate's condition.

2.6 Ammonia stripping unit: Ammonia stripping treatment was conducted every day from day 49 till day 333 (the start of the control scenario). In PHAS, the hydrolysis tanks' daily effluent was treated before entering the main digester. In SSAS, the recycling ratio, i.e., the percentage of the reactor's working volume to be treated and fed back to the digester, was initially set at 10% per day. However, an abrupt drop in biogas production was noticed, and therefore the recycling ratio was adjusted to 20% per day for the first three HRTs of the side-stream ammonia stripping scenario to facilitate the transition to a 10% recycling ratio. The decanted digestate for treatment was passed through a ⅛″ mesh to separate the solids and liquids. The liquid/solid separation step was necessary for the SSAS setup to avoid exposing methanogens to the stripping conditions, which could lead to slowing their growth. Then, the solids were returned to the digesters, and the liquids underwent ammonia stripping before being added to the reactor.

The ammonia stripping treatment for both PHAS and SSAS systems started by pre-heating the hydrolyzed PM or the decanted digestate to the stripping temperature (55° C.) using a pre-heated oven for 20 minutes, and then the pH was adjusted to 9.5 using lime (Ca(OH)2) addition; the amount of lime for the PHAS system was about 24 g lime/kg PM, whereas in SSAS the amount of lime varied between 12-30 g lime/kg PM due to changes in alkalinity and recycling ratios. The pre-heating step was essential to occur before pH adjustment as pH drops when the temperature increases (Bonmatí & Flotats, 2003). Then, the modified PM was added to two elongated glass cylinders where stripping was conducted. The temperature was controlled by circulating hot water from a water bath around the cylinder. Air (PHAS-1 and SSAS-3) and renewable natural gas (PHAS-2, SSAS-1, and SSAS-3) were used separately as stripping mediums. As an equivalent alternative to RNG, natural gas lines from the building were used for stripping, and it consisted mainly of methane (94% v/v) and ethane (5% v/v). The flow rate was set to 100 L gas/L sample/hour for three hours based on the authors' previous work (Adghim et al., 2022). The carrier gas tube was connected to the bottom of the stripping vessel. The stripped material was then fed to the main digester through a funnel.

2.7 Analytical methods: Samples were analyzed shortly after collection at any stage to ensure accurate results. TS and VS were determined using standard method No. 2540 according to APHA. VFAs were measured using the Esterification method: Hach TNT872 (50-2500 mg CH3COOH/L); COD was measured using the Reactor Digestion Method: Hach TNT822 (20-1500 mg COD/L); total alkalinity was measured using colorimetric method 10239: Hach TNT870 (25-400 mg CaCO3/L); TAN was measured by Salicylate method: Hach TNT (2-47, 100-1800 mg NH3-N/L). Biogas characterization was conducted using a 5′×0.125″ SS 100/120 HayeSep™ T column fitted in a gas chromatography instrument (GOW-MAC™ Series 400, Bethlehem, PA). Helium was used as a carrier gas, the column, detector, and injector temperatures were set to 50, 185, and 50° C., respectively, and the current was set to 50 mA.

### 3. Results and Discussion

3.1 Ammonia fermentation: Ammonia levels in the diluted PM fed to the hydrolyzer reactors were around 4225±612 mg NH3-N/L throughout the first 280 days of the experiment, after which a new batch of PM was collected from the same location. However, the diluted PM from the second batch had higher ammonia levels of 6129±485 mg NH3-N/L. Due to hydrolysis, ammonia from the first and second batches increased to 5125±540 and 7117±392 mg NH3-N/L, respectively. Such high ammonia levels increase the risk of inhibition in the digester and may cause a complete shutdown of biogas production. Therefore, ammonia stripping becomes essential to improve the digestion of PM (Y. Chen et al., 2008; Rajagopal et al., 2013; Zhuang et al., 2018). The increase in ammonia due to hydrolysis translates to final TAN/TKN ratios of 68 and 86%, leaving an average of 32 and 14% of TKN as organic nitrogen at the end of hydrolysis of batches 1 and 2, respectively. The ammonia fermentation from the first batch was lower than that observed in a previous study by Adghim et al. (2023) and Sürmeli et al. (2017), where about 88-90% of TKN consisted of ammonia after biological hydrolysis of PM. This could be due to short-circuiting in the reactor. To amend this, the hydrolysis reactors were switched to a batch mode for SSAS-1 till the end of the experiment. Switching hydrolysis to batch mode increased TAN/TKN levels from 68 to 94%. This indicates that biological hydrolysis at 40° C. was sufficient for ammonia fermentation without the need for elevated temperatures or the addition of acids or alkaline that promote a faster hydrolysis rate (Hejnfelt & Angelidaki, 2009; Yin et al., 2019).

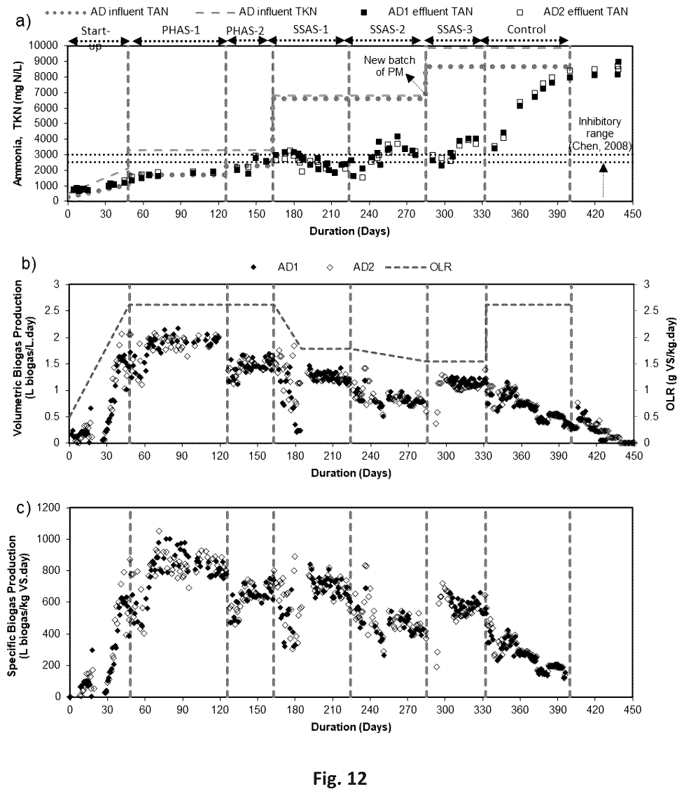

3.2 Post-hydrolysis ammonia stripping: As presented in FIG. 12a, ammonia levels in the digesters started at 710±38 mg NH3-N/L since it was only filled with digestate from the biogas plant. Before the PHAS-1 (ammonia stripping with air) scenario started, the ammonia levels increased gradually to 1068±40 mg NH3-N/L during the start-up phase with OLR increasing from 0.5 to 2.6 g kg VS·day, which was done to avoid shocking the microorganisms. At day 49, the reactors were fed with PHAS-1 feed, and ammonia levels increased to 1742±170 mg NH3-N/L over 35 days and stabilized at that level until the end of PHAS-1 (day 126). PHAS-1 consistently removed 71-75% of ammonia before the ammonia-stripped PM was fed to the main reactors, which aligns with reported values by Adghim et al. (2023) and Huang et al. (2019).

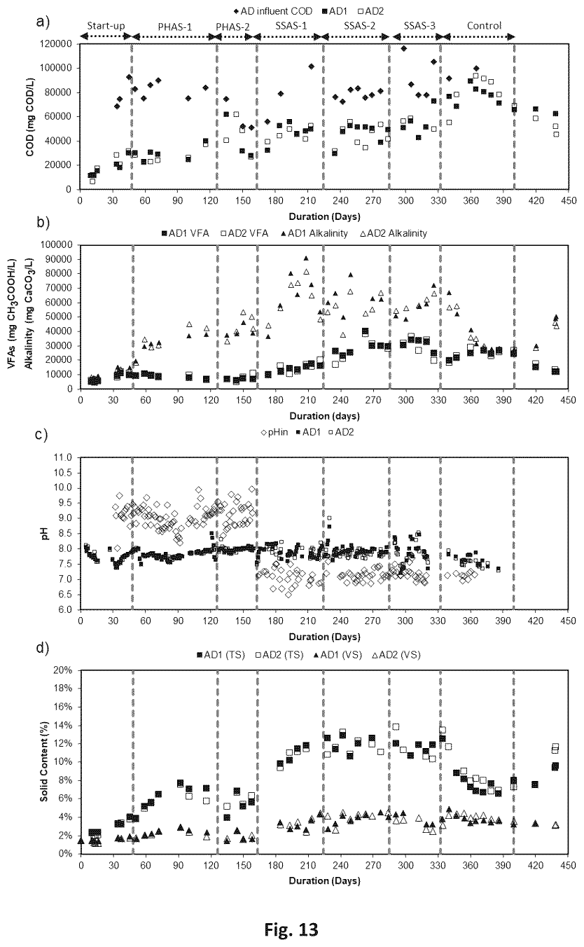

The PHAS-1 treatment led to a stable volumetric biogas production (VBP) of 1.91±0.3 L/L·day and specific biogas production (SBP) of 831±59 L biogas/kg VS·day, as shown in FIGS. 12b and 13c, with a stable average methane content of 66±2%. Such high biogas and methane production levels indicate that PHAS can reliably make the mono-digestion of PM possible. Moreover, the observed biogas production highlights the great potential of PM as a biogas feedstock. Compared with more common livestock manure used as biogas feedstock such as cow and swine manure, PM has higher organic content leading to higher biogas production. Biogas production from cow and swine manure is typically around 225 and 500 L biogas/kg VS, whereas biogas production from PM was repeatedly reported at around 700-800 L biogas/kg VS after ammonia-targeted treatments (Bi et al., 2020; Qiao et al., 2011).

FIG. 12a shows that ammonia levels in the main digesters during PHAS-1 were equivalent to the ammonia levels in their influent and remained below the widely accepted inhibitory levels (>2500 mg NH3-N/L) reported by Chen et al. (2008). The VS mass balance around the main digesters indicated that 78% of the influent VS mass was converted to biogas. No information was reported in the literature about VS removal due to PHAS treatment to compare with this study. However, the observed VS removal in this study was in line with VS removal reported by Li et al. (2018) for treating poultry manure in a SSAS system. This VS removal was also reflected in the COD removal, as 69% of the influent's total COD was destroyed (FIG. 13a). These indicators prove that PHAS-1 successfully achieved stable biogas production and alleviated the inhibitory effects of ammonia.

Despite the partial ammonia fermentation that occurred during PHAS-1 and PHAS-2 in the hydrolysis stage referred to in Section 7.3.1, the difference between the main digester's influent TAN and the effluent TKN levels indicate low to no additional ammonia fermentation in the main digester, which helped maintain the reactor's stability. The lack of additional ammonia fermentation in the main digester may indicate the lack of fermentative bacteria responsible for breaking down proteins and amino acids into ammonia.

Interestingly, the batch experiment in the study by Adghim et al. (2023) showed that VFAs were not significantly consumed during digestion, and most biogas production was conducted through the hydrogenotrophic pathway. However, in the PHAS-1 semi-continuous system, VFA levels dropped from 24636±2534 mg acetic acid/L in the feed to 8725±940 mg acetic acid/L in the digester (FIG. 13b), indicating that acetic acid consumption contributed to biogas production through acetoclastic methanogens. This indicated that the continuous ammonia stripping treatment had alleviated the ammonia inhibitory effects on acetoclastic methanogens, which was not possible in the batch mode due to the short duration of the experiment.

The pH in the AD reactors was reasonably consistent throughout the treatment phases due to the high alkalinity levels resulting from lime addition during the treatment. During the start-up stage, pH dropped from 8.1 to 7.5 and then increased to 7.7 after PHAS-1 started and stabilized for the remainder of the experiment with minimal variations, as shown in FIG. 13c.

At day 126, the ammonia stripping treatment switched to PHAS-2 (ammonia stripping with RNG) at the same tower conditions of PHAS-1, i.e., pH 9.5, 55° C., and 100 L RNG/L/hr. The ammonia removal efficiency of RNG was 50-58%, less than that of air (71-75%). Lower ammonia removal efficiency by the application of RNG increased the digestate's ammonia levels from 1742±170 mg NH3-N/L in PHAS-1 to 2354±401 mg NH3-N/L, close to the inhibitory levels (Y. Chen et al., 2008). This led to a sudden drop in SBP from 831±59 to 680±48 L biogas/kg VSadded·day (18.2% drop). The VS and COD removal consequentially reduced from 78 and 69% in PHAS-1 to 68% and 43% in PHAS-2. However, biogas production was stable and consistent throughout the PHAS-2 scenario duration. The proportionality between ammonia levels and biogas production in this semi-continuous experiment was also observed in a previous study by the authors where batch experiment comparing air versus RNG as stripping mediums was conducted (Adghim et al., 2023). Despite the decrease in biogas production, PHAS with RNG successfully maintained a VFA/ALK ratio below 0.3 as shown in FIG. 13b.

The reactors needed a short time to stabilize after transitioning from PHAS-1 to PHAS-2 (4-6 days) because the feed properties were comparable in both phases, except for the higher ammonia levels in PHAS-2. This also indicates a level of acclimation of the methanogens to the PM and partly high ammonia levels. Since the digestate characteristics and the biogas production were within 10% for over 20 days, PHAS-2 was stopped at day 162 (1.85 HRT).

3.3 Side-stream ammonia stripping: At day 163, the ammonia stripping treatment switched to side-stream ammonia stripping (SSAS), starting with RNG and then air. Initially, it was intended to treat 10% of the reactor volume per day in order to be in line with conditions previously discussed in the literature (Serna-Maza et al., 2014; Yin et al., 2019; W. Zhang et al., 2017). However, the reactors showed signs of stress during the first 10 days of ammonia stripping treatment with a 10% per day recycling ratio, and biogas production almost plummeted due to the VFA increase (FIG. 12b and FIG. 13b). Therefore, the treatment ratio was increased to 20% per day at day 154, and the OLR was reduced to 1.8 g kg VS·day (FIG. 12a) in an attempt to remedy the reactors and restore stable biogas production. The biogas production remained unstable for an additional 20 days but increased gradually, after which it stabilized at SBP of 703±60 L biogas/kg VSadded·day until the end of SSAS-1 (day 203). The biogas production was slightly higher than PHAS with RNG (PHAS-2) and less than PHAS with air (PHAS-1), +3.4% and −15.4%, respectively. Ammonia levels in the reactors were reduced from 2700 to 1900 mg NH3-N/L due to the SSAS-1 treatment, which consistently achieved similar ammonia removal efficiency as PHAS-1 (60-65%). The biogas production results show the superiority of the PHAS treatment over SSAS as PHAS achieved higher biogas production at significantly lower stripping requirements; SSAS-1 treated 20% of the reactor's working volume per day, whereas PHAS treated the incoming flow from the hydrolyzer, which was equivalent to 5% of the reactor's working volume.

Switching the feeding from ammonia-stripped PM to hydrolyzed PM led to increased VFAs in the digester due to the higher concentration of VFAs in the hydrolyzed PM than in ammonia-stripped PM. However, this increase was accompanied by an increase in alkalinity due to the higher lime influx needed for treating higher amounts in SSAS-1 than in PHAS. Therefore, the VFA/ALK was maintained below 0.3, and the stability of the reactor was not compromised (Holliger et al., 2016).

The SSAS-1 treatment required almost double the amount of lime to raise the digestate's pH to 9.5 compared with PHAS because the treated amount of digestate was higher in SSAS-1. However, both systems required the same lime dosage per volume (18 g lime per kg digestate). As a result, the TS and the VS in the AD reactors steadily increased from 6.3 to 11.6% and from 2.6 to 4.25%, respectively (FIG. 13d). A similar increase in TS % was reported by Zhang et al. (2017). The addition of inorganic lime cannot justify the increase in VS %, raising concerns about the possibility of unintended sedimentation at the bottom of the reactor during the PHAS scenarios. To verify the significance of sedimentation in the reactors, the contents of the reactors were decanted at day 436 (the end of the experiment), and the solids at the bottom of the reactors were scraped and added to the rest of the reactor contents. If sedimentation were significant throughout the experiment, the TS measurement would be significantly higher than 9-10% (the feed's total solids). However, the TS in AD1 and AD2 were 9.5 and 11.5%, respectively, indicating no significant settlement throughout the experiment. The treatment of 20% of the reactor's volume at 55° C. for three hours every day may have contributed to increasing the total and volatile solids content.

In large-scale plants, removing 20% of the reactor's working volume per day for 2-3 hours for treatment would disrupt the operation (K. Li et al., 2018; Serna-Maza et al., 2014; W. Zhang et al., 2017) and lead to large treatment units. Therefore, after the performance of the AD reactors was stabilized with SSAS-1 conditions, the recycling ratio was dropped to 10% per day (SSAS-2) while maintaining OLR and other stripping tower conditions the same, i.e., pH 9.5, 55° C., and RNG flowrate of 100 L RNG/L digestate/hour, in order to assess the stability of the reactor at lower treatment proportions.

SSAS-2 remained operational from day 243 to day 303 (3 HRTs). The impact of reducing the treatment proportion was reflected in the biogas production and ammonia levels almost immediately after the treatment was switched to a 10% recycling ratio. The VBP and SBP dropped from 1.16 L/L/day in SSAS-1 to 0.88 L/L/day in SSAS-2 and from 703±60 L biogas/kg VSadded in SSAS-1 to 475±40 L biogas/kg VSadded in SSAS-2, respectively. These drops were mainly due to ammonia levels increasing rapidly from 1900 to 3186±218 mg NH3-N/L. Having said that, the SSAS-2 treatment successfully maintained stable continuous operation, despite being under sub-optimal conditions. The methane content in biogas was reduced from around 65% in SSAS-1 to 58% in SSAS-2.

These sub-optimal conditions of SSAS-2 also led to VFA accumulation at around 29722±1199 mg acetic acid/L, which increased from 14086±1843 mg acetic acid/L in SSAS-1. Also, due to the reduced amount of lime added for the treatment, the alkalinity dropped from 66856 to 55622 mg CaCO₃/L. As a result, the VFA/ALK ratio increased to 0.51, above the recommended 0.3 ratio (Nguyen et al., 2019; Reyes et al., 2015). However, there was no apparent effect of these challenges on the pH of the effluent, as it was stabilized at 7.9 throughout the experiment (FIG. 13c). This pH level is favorable for methanogenic archaea and can reduce the competition over carbon with sulfur-reducing or sulfur-oxidizing bacteria since these types of bacteria often prefer lower PH levels, which explains why the drop in biogas production, as well as methane content, was not drastic (Fotidis et al., 2014; Rajagopal et al., 2013).

The ammonia removal efficiency in SSAS-2 did not differ from SSAS-1 since they were conducted at similar tower conditions (both achieved 60-65% ammonia removal). However, ammonia levels increased in the main digesters due to the lower recycling ratio, which eventually led to higher ammonia levels in the ammonia stripping unit effluent in SSAS-2 than SSAS-1 (1050 mg NH3-N/L in SSAS-2 compared to 600 mg NH3-N/L in SSAS-1).

The last treatment (SSAS-3) represented the use of air in side-stream ammonia stripping at a 10% recycling ratio and under similar stripping tower conditions, i.e., pH 9.5, 55° C., and flowrate of 100 L air/L digestate/hour. Recently, the use of air in SSAS has been of interest due to its possibility to achieve higher ammonia removal efficiencies in a shorter time compared with other gases and its lower impact on the digestate's buffer capacity (Bousek et al., 2016).

A new sample was collected from the farm for this part of the experiment. Fortunately, the collection time and conditions were similar to the first batch, which minimized the differences in the manure's characteristics, specifically the moisture content. Moreover, the farm also confirmed that no diet or operations changes occurred that could significantly affect the quality of manure. However, ammonia levels in the second batch were higher than in the first batch, increasing ammonia levels during SSAS-3 despite achieving higher ammonia removal efficiency (70±3%) than SSAS-2 (FIG. 12a). As a result, the ammonia levels of hydrolyzed PM from the second batch reached 8100 mg NH3-N/L, compared to 6000 mg NH3-N/L from the first batch.

Despite the slight increase of the digestate's ammonia levels during SSAS-3, VBP and SBP increased to 1.14 L biogas/L/day and 565±43 L biogas/kg VSadded/day, respectively. The increase in biogas production was accompanied by a significant drop in VFAs from 31720 to 20694 mg CH3COOH/L. The reactors' alkalinity in SSAS-3 remained similar to SSAS-2 (58568 mg CaCO3/L) because the lime dosage and treatment proportions remained the same. As a result, VFA/ALK reduced to 0.35, providing a more stable operation than observed in SSAS-2 when RNG was used. This indicates that the second batch of poultry manure may have been more readily biodegradable than the first batch, which could be evident by observing the improvement in VFA consumption.

One of the concerns when using air for ammonia stripping, especially for SSAS systems, is that it could lead to the toxification of methanogens when the treated portion is fed back to the reactor. However, the high biogas production observed in SSAS-3 conducted in this study shows no evidence of toxification or inhibition due to using air. Moreover, dissolved oxygen (DO) was measured immediately after stripping with air and was found close to 2.5-3 mg/L and declined rapidly (within 20 minutes) to below 0.2 mg/L. A similar conclusion was also observed by (Fernandez-Gonzalez et al., 2019), where it was reported that even with no liquid/solid separation, air SSAS did not negatively impact biogas production. However, it shifted the microbial presence towards hydrogenotrophic methanogens.

It should be noted the recycling ratio in this study was higher than in previous SSAS studies discussing food waste (Serna-Maza et al., 2014; W. Zhang et al., 2017). Having said that, there are some key differences between the current study and the above studies that compelled the higher recycling ratio in this study. First, most SSAS studies discussed thermophilic digesters with temperatures set to 55-60° C., whereas this study discussed mesophilic digesters. Moreover, treatment conditions, including recycling ratio and stripping tower conditions, depend on the feedstock's ammonia levels and removal efficiency. Poultry manure in this study had ammonia levels reaching 6000-8100 mg NH3-N/L before treatment. On the other hand, the food waste ammonia levels discussed in Serna-Maza et al. (2014) and Zhang et al. (2017) were limited to 4000-6000 mg NH3-N/L, allowing less recycling ratios to be effective.

3.4 Control scenario: The last phase of this study was to assess the reactors' performance when no ammonia stripping treatment was applied at any stage. At day 333, the reactors were fed with hydrolyzed PM characterized by high ammonia levels nearing 6000-8100 mg NH3-N/L at the initial OLR (2.6 g kg VS·day). As a result, the reactors' biogas production started declining steadily and stabilized at VBP 0.524 L biogas/L digestate/day and SBP of 154±20 L biogas/kg VS/day. The SBP during the control scenario was 81, 77, 78, 68, and 73% less than PHAS-1, PHAS-2, SSAS-1, SSAS-2, and SSAS-3, respectively. Moreover, the methane content dropped to 40% of the total biogas production, whereas it was stable at 58-65% during the previous phases. In addition, the VS removal dropped from 52-78% in previous phases to 17% in the control scenario, indicating clear signs of inhibition. Interestingly, the drop in biogas production was not abrupt, indicating some degree of acclimatization of methanogens to high ammonia levels.

The drop in biogas production due to high ammonia levels was accompanied by increased VFA and COD levels of the reactors' effluents. In addition, due to the discontinued lime addition in this phase, alkalinity dropped from around 70000 to 28000 mg CaCO₃/L, equivalent to the alkalinity levels of the hydrolyzed PM fed to the reactor. As a result, VFA/ALK ratio increased to around 0.9, leading to a drop in pH to 7.46±0.08, which is still favorable for methanogenic microorganisms. However, with ammonia levels rapidly increasing to 8100±100 mg NH3-N/L, it was clear that the process was inhibited and operated under sub-optimal conditions. TKN levels also increased significantly to 10111 mg TKN-N/L by the end of the control scenario.

After day 400, the feeding stopped. The reactors were monitored for biogas production and other characteristics. Biogas production gradually dropped to 0.05 L biogas/L/day. Only a few properties were different from the end of the control scenario. Due to the prolonged period after feeding, it was noticed that COD and VFA concentrations decreased to 53206 mg COD/L and 12000 mg CH₃COOH/L, respectively, due to the continuation of biogas production. The sudden increase in alkalinity and TS at the end of the experiment was due to scraping the solids precipitated at the bottom, which included some of the lime particles that were added during the treatments.

The control scenario results showed that the mono-digestion of poultry manure is not feasible without further treatment. Treatment with PHAS and SSAS improved biogas production by 5.3- and 4.5-fold, respectively. In addition, other performance parameters, such as ammonia, VFA/ALK, and VS removal, were all improved significantly due to the stripping treatments.

In Adghim et al. (2023), hydrolyzed PM resulted in about 340 L CH4/kg VS in the batch mode. However, ammonia levels were capped at 3300 mg NH3-N/L because the accumulation of ammonia was not possible. On the other hand, the continuous feeding of the reactors in the current study allows for ammonia accumulation, which led to methane production of only 61 L CH4/kg VS/day at ammonia levels of 8100 mg NH3-N/L.

### 3.5 Discussion of Results

Discussion on post-hydrolysis versus side-stream ammonia stripping: All treatments in this study showed that mitigating ammonia inhibition and allowing the mono-digestion of poultry manure is feasible at different degrees. For instance, PHAS systems treated all incoming effluent from the hydrolyzer (500 ml/day per reactor), whereas SSAS treated higher volumes of the digestate (1000-2000 ml/day per reactor) to compete with the performance of PHAS systems. However, even at these high recycling ratios, PHAS outperformed SSAS and maintained a more stable operation of the reactors in terms of biogas production and digestate characteristics (680-830 L biogas/kg VS·day versus 475-701 L biogas/kg VS·day). Moreover, PHAS successfully alleviated ammonia inhibitory effects at a higher OLR than SSAS (2.6 and 1.8, respectively).

Despite the advantages of PHAS, there are some situations where the use of PHAS is not applicable. One example is in biogas plants operating a one-stage AD configuration, where building a side-stream ammonia stripping may be more logical than building an additional reactor for hydrolysis intended specifically to allow PHAS, which would require additional space, operation and maintenance, and capital costs.

Despite the two systems having different approaches, some mirroring effects were observed when using air or RNG, where air treatment always led to higher biogas production. However, using air in biogas plants may be unfavorable for several reasons, such as avoiding the need to implement a solid/liquid separation prior to the treatment in the SSAS case or the risk of air infiltration to the main digester. Therefore, it was essential to find an alternative carrier gas that could achieve similar ammonia removal efficiency of air, which RNG proved successful at.

3.6 Discussion on selected parameters for side-stream ammonia stripping: This part of the discussion addresses the significant differences between the stripping conditions conducted throughout the SSAS in this study and those more common in the literature. The determination of the stripping tower conditions depends mostly on the type of carrier gas used. For example, Serna-Maza et al. (2014) and Zhang et al. (2017) used biogas for ammonia removal, which can significantly reduce the pH of the solution due to its high CO2 content if pumped at high flowrates. Therefore, in all these studies, biogas flowrate is often limited between 1-10 L biogas/L digestate/hour, the temperatures were set to 65-75° C., pH was increased above 10, and the duration of stripping ranged between 1-3 days per treatment batch. The high temperature and pH translate to high energy and material demands that can be burdensome to the plant.

Alternatively, using air in Fernandez-Gonzalez et al. (2019) and RNG and air in this study, higher ammonia removal efficiency was achievable at significantly lower pH and temperature requirements than biogas recirculation. Moreover, if ammonia stripping columns were operated in batch or semi-batch modes, the required hours for treatment using air or RNG compared with biogas would be 17-21 hours per week versus 168 hours per week with biogas, respectively. This means that systems with an efficient carrier gas will require significantly less energy and alkaline materials to achieve high ammonia removal efficiency, whether in PHAS or SSAS. Having said that, the carrier gas flowrate using air or RNG was almost 5-10 times the flowrate reported for biogas stripping. This may pose a challenge in finding suitable and affordable air or RNG pumps or using multiple pumps per stripping column. There is a clear trade-off between the amount of energy and material (lime) needed to achieve high ammonia removal efficiency and the carrier gas flow rate, regardless of whether it is a PHAS or a SSAS system.

3.7 Discussion on air versus RNG as stripping mediums: Ammonia stripping with RNG is a new concept, and it shows promising results regarding alleviating ammonia inhibition and achieving stable biogas production. Compared with air, RNG has lower ammonia removal efficiency and consequentially less improvement in biogas production. However, when compared with the control (no treatment) scenario, which was inhibited due to high ammonia levels, it is clear that RNG is a viable stripping medium. Moreover, in situations where anaerobic conditions must be maintained during stripping, RNG can be more advantageous than air due to its high purity (>98% CH₄) preventing risk of oxygen toxicity. Having said that, stripping conditions may need to be modified to achieve higher ammonia removal efficiency. Moreover, RNG can offer other advantages as an efficient anaerobic stripping alternative to the widely discussed stripping gases in literature, i.e., biogas, steam, or nitrogen. Additionally, unlike nitrogen, RNG is available in-situ and can achieve higher ammonia removal efficiency than biogas and water steam under significantly gentler conditions, i.e., lower pH, temperature, and duration of treatment.

4. Conclusions: This study investigated the performance of the three possible ammonia stripping configurations intended to reduce ammonia levels in poultry manure to improve its methane potential based on experimental results. Amongst the three ammonia stripping approaches, post-hydrolysis ammonia stripping was the most advantageous and flexible configuration as it resulted in the highest biogas production and maintained sub-inhibitory levels with more efficiency than side-stream and pre-hydrolysis ammonia stripping. Side-stream ammonia stripping also proved successful in achieving stable biogas production, however, it required more treatment to compete with post-hydrolysis ammonia stripping.

### REFERENCES

Abouelenien et al., Bioresource Technology (2010) 101:6368-6373
Adghim et al., Bioresource Technology Reports (2023) 21:101334
Adghim M. et al., Journal of Environmental Management (2022) 319:115717
Adghim M. et al., Waste and Biomass Valorization (2021) 12:6045-6056
Agyeman et al., Journal of Environmental Management (2014) 133:268-274.
Baldi et al., Sustainability (Switzerland) (2018) 10 (9): 3073
Bi et al., Renewable Energy (2020) 146:2380-2389
Bonmati and Flotats., Waste Management (2003) 23:261-272
Botheju et al., Modelling, Identification and Control (2011) 31 (2): 55-65
Bousek et al., Bioresource Technology (2016) 203:259-266
Chen et al., Bioresource Technology (2008) 99 (10): 4044-4064
Chen et al., Chemical Engineering Science (2021) 237:116534
Fakkaew and Polprasert., Bioresource Technology Reports (2021) 14:100647
Fernandez-Gonzalez et al., Microbial Biotechnology (2019) 12 (6): 1403-1416
Fisher et al., Critical Reviews in Environmental Science and Technology (2019) 49(17): 1515-1586
Fotidis et al., Environmental Science and Technology (2014) 48 (13): 7669-7676
Fuchs et al., Renewable and Sustainable Energy Reviews (2018) 97:186-199
Hejnfelt & Angelidaki., Biomass and Bioenergy (2009) 33 (8): 1046-1054
Holliger et al., Water Science and Technology (2016) 74 (11): 2515-2522
Huang et al., Biomass and Bioenergy (2016) 90:139-147
Huang et al., International Bio deterioration and Biodegradation (2019) 140:84-89
Li et al., Fuel (2018) 232:395-404
Meegoda et al., Journal of Environmental Research and Public Health (2018) 15(10): 1-16
Nasir et al., Appl Microbiol Biotechnol (2012) 95:321-329
Nguyen et al., Bioresource Technology (2019) 280:269-276.
Nie et al., Bioresource Technology (2015) 178:238-246
Nielsen et al., Biomass and Bioenergy (2013) 50:10-18
Pan et al., International Journal of Hydrogen Energy (2013) 38 (29): 12747-12754
Qiao et al., Renewable Energy (2011) 36 (12): 3313-3318
Rajagopal et al., Bioresource Technology (2013) 143:632-641
Reyes et al., Journal of Environmental Chemical Engineering (2021) 9 (1): 104695
Romero-Guiza et al., Biomass and Bioenergy (2014) 67:243-251
Serna-Maza et al., Bioresource Technology (2014) 152:307-315
Surmeli et al., Water Science and Technology (2017) 75 (12): 2811-2817
Usack et al., Journal of Visualized Experiments (2012) 65:1-9
Usack et al., Water Research (2015) 87:446-457
Walker et al., Chemical Engineering Journal (2011) 178:138-145
Yin et al., Bioresource Technology (2019) 287:121470
Zhang et al., Bioresource Technology (2017) 244:611-620
Zhang et al., Journal of Hazardous Materials (2012) 199-200:36-42

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “a unit” includes one or more of such units and reference to “the process” includes reference to equivalent steps and methods known to those of ordinary skill in the art that could be modified or substituted for the processes described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses, and such.

It is understood that the examples and embodiments described herein are for illustrative purposes only and that various modifications or changes in light thereof will be suggested to persons skilled in the art and are to be included within the present invention and scope of the appended claims.

1. A continuous or semi-continuous two-stage anaerobic digestion process for converting organic material into biogas, comprising the steps of:
(1) hydrolysing the organic material under anaerobic conditions in a first-stage anaerobic digestion unit to obtain hydrolyzed material;
(2) stripping ammonia from the hydrolyzed material in a NH3-stripping unit to obtain an ammonia-stripped slurry;
(3) proceeding to methanogenesis of the ammonia-stripped slurry in a second-stage anaerobic digestion unit to obtain a digestate and biogas;
wherein said process operates in a continuous or semi-continuous mode.

2. The process of claim 1, wherein operation in a continuous or semi-continuous mode comprises at least one of: (i) feeding organic material to the first-stage anaerobic digestion unit one or more times per day; (ii) drawing hydrolyzed material from the first-stage anaerobic digestion unit one or more times per day; (iii) drawing some of the ammonia-stripped slurry one or more times per day; and (iv) drawing a digestate from the second-stage anaerobic digestion unit one or more times per day.

3. The process of claim 1, wherein said stripping is carried out at a constant temperature of about 55° C.

4. The process of claim 1, wherein said stripping is carried out at a pH about 9.5.

5. A continuous or semi-continuous two-stage anaerobic digestion system for converting organic materials into biogas, comprising:
a first-stage anaerobic digestion unit configured to hydrolyse the organic material under anaerobic conditions and to obtain hydrolyzed material;
a NH₃-stripping unit configured to strip ammonia from the hydrolyzed material and obtain an ammonia-stripped digestate;
a second-stage anaerobic digestion unit configured for methanogenesis of the ammonia-stripped digestate and obtain a second digestate and a biogas comprising methane biogas;
wherein said system is adapted for operation in a continuous or semi-continuous mode.

6. The system of claim 5, wherein the system comprises at least one of (i) a first controller for controlling a loading rate of organic material at step (1); (ii) a second controller for controlling a loading rate of slurry hydrolyzed material at step (2); (iii) a third controller for controlling a content of ammonia-stripped slurry and/; (iv) a forth controller for controlling an amount of digestate at step (3).

7. A two-stage anaerobic digestion process for converting organic materials into biogas, comprising the steps of:
(1) hydrolysing the organic material under anaerobic conditions in a first-stage anaerobic digestion unit to obtain hydrolyzed material;
(2) stripping ammonia from the hydrolyzed material in a NH₃-stripping unit to obtain an ammonia-stripped digestate, wherein said stripping comprises stripping with upgraded biogas;
(3) proceeding to methanogenesis of the ammonia-stripped digestate in a second-stage anaerobic digestion unit comprising to obtain a second digestate and a biogas comprising methane biogas.

8. The process of claim 7, further comprising collecting the biogas of step (3) and feeding at least a portion thereof to step (2).

9. The process of claim 7,
wherein said biogas comprises carbon dioxide, and
wherein the process further comprises the steps of:
(4) removing carbon dioxide from the biogas of step (3) to obtain upgraded biogas;
(5) feeding said upgraded biogas to step (2).

10. The process of claim 7, wherein the upgraded biogas comprises biomethane.

11. The process of claim 7, wherein said stripping is entirely anaerobic.

12. A two-stage anaerobic digestion system for converting organic materials into biogas, comprising:
a first-stage anaerobic digestion unit configured to hydrolyse the organic material under anaerobic conditions and obtain hydrolyzed material;
a NH₃-stripping unit configured for stripping ammonia from the hydrolyzed material and obtaining an ammonia-stripped digestate, wherein said NH₃-stripping unit is configured for stripping with upgraded biogas;
a second-stage anaerobic digestion unit configured for methanogenesis of the ammonia-stripped digestate and obtain a second digestate and a biogas.

13. The system of claim 12, wherein said biogas comprises carbon dioxide, and wherein the system further comprises a biogas collector configured for collecting said biogas and removing carbon dioxide therefrom and obtain an upgraded biogas.

14. The system of claim 13, wherein said system is configured for feeding said upgraded biogas to the NH₃-stripping unit, and wherein said system is adapted for the stripping of ammonia with the upgraded biogas obtained from the biogas collector.

15. A two-stage anaerobic digestion process for converting organic materials into biogas, comprising the steps of:
(1) hydrolysing the organic material under anaerobic conditions in a first-stage anaerobic digestion unit to obtain hydrolyzed material;
(2) proceeding to methanogenesis of the hydrolyzed material in a second-stage anaerobic digestion unit to obtain a digestate and a biogas comprising methane biogas;
(3) stripping ammonia from the digestate in a NH₃-stripping unit to obtain an ammonia-stripped digestate, wherein said stripping comprises stripping with upgraded biogas.

16. The process of claim 15, further comprising collecting the biogas of step (2) and feeding at least a portion thereof to step (3).

17. The process of claim 15,
wherein said biogas comprises carbon dioxide, and
wherein the process further comprises the steps of:
(4) removing carbon dioxide from the biogas of step (2) to obtain upgraded biogas;
(5) feeding said upgraded biogas to step (3).

18. A two-stage anaerobic digestion system for converting organic materials into biogas, comprising:
a first-stage anaerobic digestion unit configured to hydrolyse the organic material under anaerobic conditions and obtain hydrolyzed material;
a second-stage anaerobic digestion unit configured for methanogenesis of the hydrolyzed material and obtain a second digestate and a biogas; and
a NH₃-stripping unit configured for stripping ammonia from the second digestate and for obtaining an ammonia-stripped digestate;
wherein said NH₃-stripping unit is further configured for stripping with upgraded biogas.

19. The system of claim 18, wherein said biogas comprises carbon dioxide, and wherein the system further comprises a biogas collector configured for collecting said biogas and removing carbon dioxide therefrom and obtain an upgraded biogas.

20. The system of claim 19, wherein said system is configured for feeding said upgraded biogas to the NH₃-stripping unit, and wherein said system is adapted for the stripping of ammonia with the upgraded biogas obtained from the biogas collector.
